(19) United States
(12) Patent Application Publication
(10) Pub. No.: US 2024/0194342 A1
(43) Pub. Date: Jun. 13, 2024

(54) Methods and Systems for Providing Medical Insights

(71) Applicant: Function Health Inc., Austin, TX (US)

(72) Inventors: Pranitha Bhimanagouda Patil, College Station, TX (US); Michael Ryan Nemke, Austin, TX (US); Jonathan Asher Swerdlin, Austin, TX (US); Daniel Brian Swerdlin, San Francisco, CA (US)

(21) Appl. No.: 18/525,158

(22) Filed: Nov. 30, 2023

Related U.S. Application Data
(60) Provisional application No. 63/431,312, filed on Dec. 8, 2022.

(51) Int. Cl. G16H 50/20; G16H 50/70

(52) U.S. Cl. CPC G16H 50/20; G16H 50/70

(57) ABSTRACT

This application is directed to managing data. A computer system constructs a digital model of a user including a plurality of features representing demographic, medical, and activity data of the user. The computer system applies a classifier model to the digital model to identify a first set of actionable insights and identifies one or more clustering models generated from user data for a plurality of users in a vector space having dimensions correspond to the plurality of features. The computer system assigns the digital model to a respective cluster within each of the one or more clustering models, and identifies a second set of actionable insights according to respective clusters assigned to the digital model within the one or more clustering models. A subset of the first and second sets of actionable insights is presented to the user on a user interface.

### RELATED APPLICATION

This application claims priority to U.S. Provisional Patent Application No. 63/431,312, filed Dec. 8, 2022, titled “Methods and Systems for Providing Medical Insights,” which is incorporated by reference in its entirety.

### TECHNICAL FIELD

The disclosed embodiments relate generally to data processing, including, but not limited to, methods, systems, and non-transitory computer-readable storage media for integrating medical data from a plurality of distinct modalities to provide supplement medical information and determine actionable insights.

### BACKGROUND

Medical doctors have treated patients with specific illnesses and conditions by reviewing medical records of their patients and requisitioning additional lab tests. Each doctor normally keeps his own medical record for a patient, and the patient does not have control over what is kept in the medical record. Even if the medical record is accessible to the patient, the medical record is organized and presented from the perspective of the doctor or hospital, and fails to provide a comprehensive view of the patient's health history according to the patient's personalized context and preferences. These limitations underscore a need for a more patient-centric approach to healthcare data management and decision-making.

### SUMMARY

Various embodiments of this application are directed to methods, systems, and non-transitory computer-readable storage media for machine learning based data processing and management of a user's private data. A digital model is constructed for the user and includes a plurality of features representing demographic, medical, and activity data of the user. Examples of these features include medical characteristics specified by the user, medical characteristics specified in a medical record and retrieved from a database of a medical institution, activity data collected by one or more wearable devices, biomarker data measured according to one or more laboratory tests of bodily fluids, and/or changes over time in any of the user-specified medical characteristics, the retrieved medical records, the activity data, and the biomarker data. The system applies a plurality of classifier models to the digital model to identify a first set of actionable insights. In a vector space whose dimensions correspond to the plurality of features, the system applies a plurality of clustering models generated from data for a plurality of users, thereby assigning the digital model to a respective cluster within each of the clustering models, and identifies a second set of actionable insights according to the assigned clusters. The system then presents the first and second sets of actionable insights to the user on a user interface. By these means, a wide range of the user's private data is analyzed using various machine learning solutions, each with its distinct focus, to generate actionable insights from diverse perspectives. This approach enhances the quality of recommendations, tailoring them more effectively to the unique conditions and preferences of the user.

In one aspect, a method is implemented to manage data at a computer system having one or more processors and memory. The method includes constructing a digital model of a user including a plurality of features representing demographic, medical, and activity data of the user. The method further includes applying a classifier model to the digital model to identify a first set of actionable insights and identifying one or more clustering models generated from user data for a plurality of users in a vector space having dimensions correspond to the plurality of features. The method further includes assigning the digital model to a respective cluster within each of the one or more clustering models and identifying a second set of actionable insights according to respective clusters assigned to the digital model within the one or more clustering models.

Some embodiments provide a means for collecting, integrating, and storing both health data and medical record data in a particular way so that the data can be used (1) by the machine-learning engine to create and update a constellation environment, and (2) by the web engine to provide the UI features, such as an “out of range” widget.

Some embodiments provide a means for analyzing the stored data to identify meaningful changes to the user profile. In some embodiments, this is a rules-based algorithm.

Some embodiments provide a “constellation environment,” where each individual is a point in the constellation. Data from each individual user is helpful to the knowledge of the constellation, and data in the constellation is useful to each individual. In some embodiments there is a specially optimized machine-learning module that creates and runs the constellation environment so that the system serves the users, and the users serve the system.

Some embodiments provide a GUI environment (e.g., accessible from a smart-phone or smart-device). The graphical user interface utilizes a web engine (e.g., a web-based server), which creates displays and widgets based on static data processed by the algorithms above and from the dynamic data processed by the machine-learning module noted above.

Thus methods, systems, and graphical user interfaces are disclosed that provide a digital hub, integrating a user's biomarker results, medical records and documents, application programming interface data, purchase history, and self-reported data, as well as data from an electronic knowledgebase of health-related data to track, display, and monitor the user's daily health data, physical activity, and internal inputs and outputs against the user's individual historical data and the data from an electronic knowledgebase of health-related data in order to provide insights, suggestions, recommendations, support, motivation, and direction to advice and care, all of which is targeted to facilitate behaviors associated with both immediate and long-term health and wellness.

Other objects, advantages, novel features, and further scope of applicability of the present disclosure will be set forth in part in the detailed description to follow, and in part will become apparent to those skilled in the art upon examination of the following or may be learned by practice of the disclosure. Although the description above contains many specificities, these should not be construed as limiting the scope of the disclosure but as merely providing illustrations of some of the presently preferred embodiments of the disclosure. As such, various other embodiments are possible within its scope. Accordingly, the scope of the disclosure should be determined not by the embodiments illustrated, but by the appended claims and their equivalents.

### BRIEF DESCRIPTION OF THE DRAWINGS

For a better understanding of the aforementioned systems, methods, and graphical user interfaces, as well as additional systems, methods, and graphical user interfaces, reference should be made to the Description of Embodiments below, in conjunction with the following drawings and appendices.

FIG. 1 is a block diagram illustrating an example network architecture that enables a multi-user data management system, in accordance with some embodiments;

FIG. 2 is a block diagram illustrating a representative computer system, in accordance with some embodiments;

FIG. 3A is a block diagram of an example insight generator of a data processing system that generates actionable recommendations, in accordance with some embodiments;

FIG. 3B illustrates user interfaces displaying actionable recommendations 130, in accordance with some embodiments;

FIG. 4A is a schematic diagram of a single-class classifier applied for binary classification, in accordance with some embodiments;

FIG. 4B is a schematic diagram of a multi-class classifier applied for multi-class classification, in accordance with some embodiments;

FIGS. 5A and 5B are block diagrams of two example classifier subsystems for providing a first set of actionable recommendations, in accordance with some embodiments;

FIGS. 5C and 5D are block diagrams of two example classifier models for providing a first set of actionable recommendations, in accordance with some embodiments;

FIGS. 6A-6D illustrate four example clustering models, in accordance with some embodiments;

FIG. 7A illustrates an example single cluster system to which a digital model of a patient user is assigned using associated features, in accordance with some embodiments;

FIG. 7B illustrates two example cluster systems to which a digital model of a patient user is assigned using two distinct subsets of features, in accordance with some embodiments;

FIG. 8 is an example user interface of a medical data application (e.g., a health insight application), in accordance with some embodiments;

FIG. 9 is a flowchart depicting a process for managing user data in accordance with some embodiments;

FIG. 10 is a flowchart depicting a more-detailed process for managing user data, in accordance with some embodiments; and

FIG. 11 is a flowchart depicting a process for generating actionable insights in accordance with some embodiments.

Reference will now be made to embodiments, examples of which are illustrated in the accompanying drawings. In the following description, numerous specific details are set forth in order to provide a thorough understanding of the present disclosure. However, it will be apparent to one of ordinary skill in the art that the present disclosure may be practiced without requiring these specific details.

Corresponding reference characters indicate corresponding components throughout the several figures of the drawings. Elements in the several figures are illustrated for simplicity and clarity and have not necessarily been drawn to scale. For example, the dimensions of some of the elements in the figures might be emphasized relative to other elements for facilitating understanding of the various presently disclosed embodiments. In addition, common, but well-understood, elements that are useful or necessary in a commercially feasible embodiment are often not depicted in order to facilitate a less obstructed view of these various embodiments of the present disclosure.

### DESCRIPTION OF EMBODIMENTS

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of claims and the subject matter may be practiced without these specific details. For example, it will be apparent to one of ordinary skill in the art that the subject matter presented herein can be implemented on many types of computer systems with data processing capabilities.

In many embodiments, as described herein, a process, system, method, and/or device can be configured to generate various actionable insights for a user. As those skilled in the art will recognize, an actionable insight may vary based on the types of laws, regulations, rules, or other limitations placed within a jurisdiction. These controlling jurisdictions may be related to either the location of the user, and/or the location of the processing of the data necessary to generate the actionable insight. In some embodiments, the actionable insight may be in the form of a recommendation. However, in certain locations, the actionable insights may be data formatted or presented for consumption by a user through one or more presentations, such as a graphical user interface or the like. Those skilled in the art will recognize that actionable insights can be utilized in a variety of ways and vary in format and generation based on the local customs, controlling laws, and/or mode of presentation. While various embodiments described herein are described as presenting actionable recommendations, it is contemplated that a broader actionable insight may be utilized as well based on the limitations set in place by one or more jurisdictions.

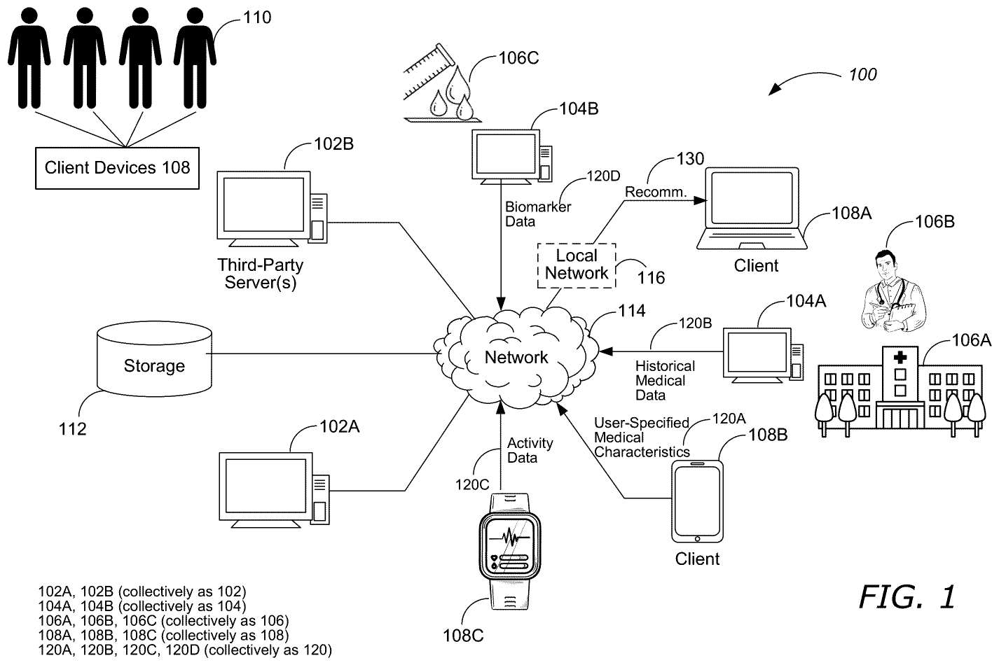

As will be described below in more detail, in certain embodiments, the demographic, medical, and activity data 120 (as depicted below in the discussion of FIG. 1) of the user 110 includes a subset of: user-specified medical characteristics 120A of the user 110; historical medical data 120B of the user 110, retrieved from a database of medical records of a medical institution 106; activity data 120C from one or more wearable devices worn by the user 110; biomarker data 120D measured by one or more laboratory tests of bodily fluids from the user 110; and changes over time (F(t)) in the user-specified medical characteristics 120A, the historical medical data 120B, the activity data 120C, and the biomarker data 120D.

In some embodiments, the digital model 242 has a predefined data structure in which the plurality of features 305 is organized to be used as inputs to the classifier models 302 and the one or more clustering models 304.

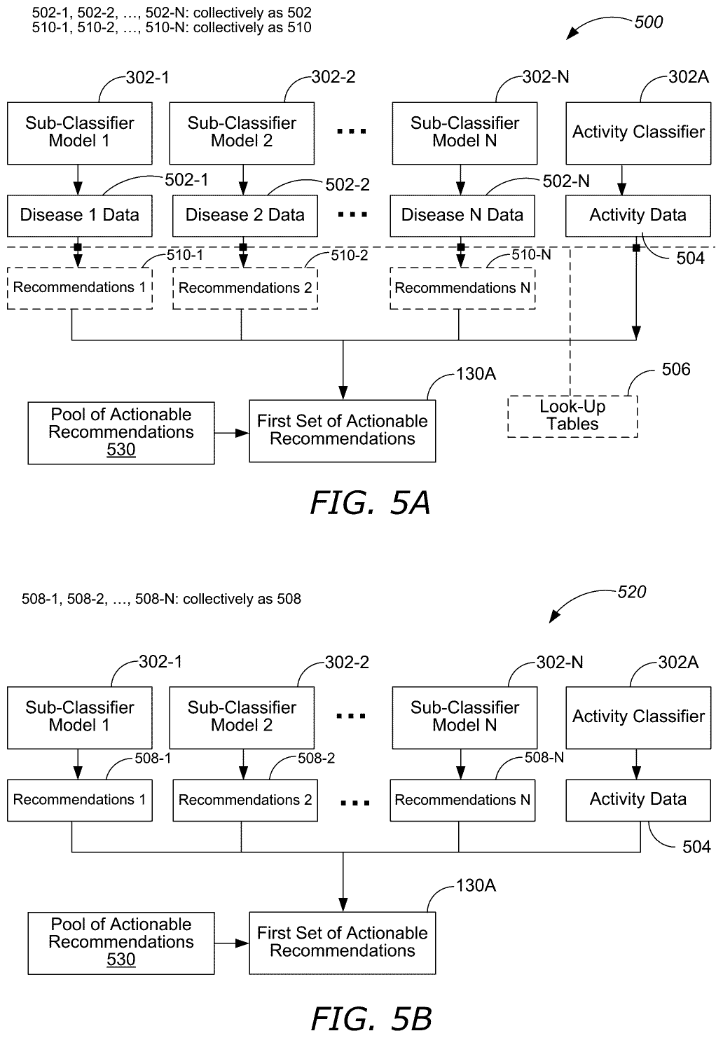

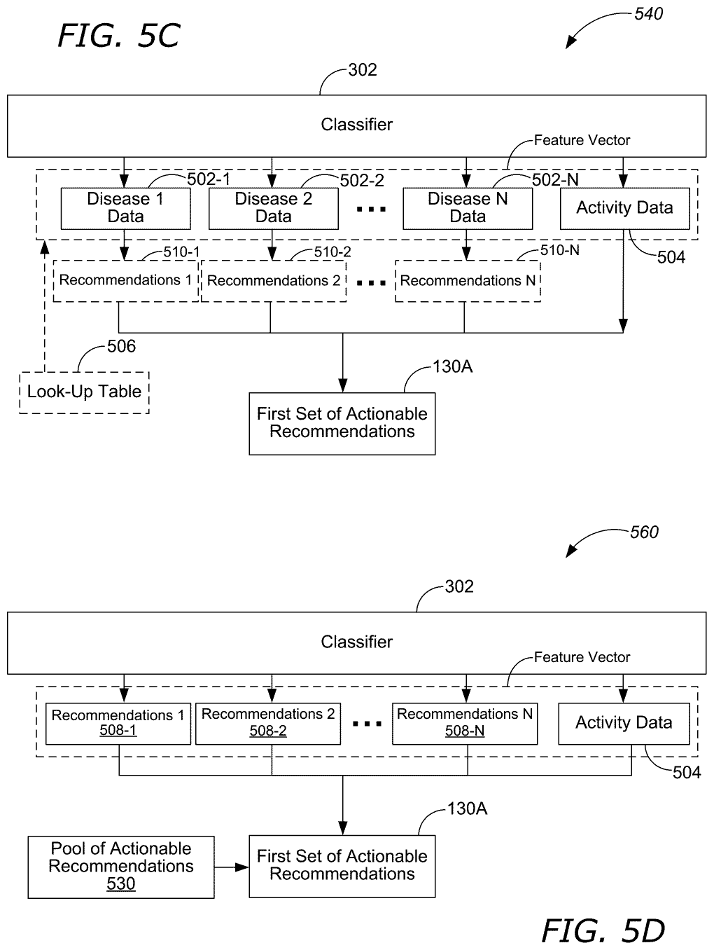

In some embodiments, the classifier models 302 include one or both of a plurality of first sub-classifier models 302-1 to 302-N and an activity classifier model 302 (as depicted below in the discussion of FIG. 5A). Each first sub-classifier model 302 is configured to determine respective condition data 502 of a plurality of predefined medical conditions 306. The activity classifier model 302 is configured to determine an activity style and an activity level (e.g., activity data 504) of the user 110.

In some embodiments, the computer system 200 applies the classifier models 302 to the digital model 242 to identify the first set of actionable recommendations 130A by generating a condition vector having a plurality of probability values corresponding to a plurality of predefined medical conditions 306 and determining the first set of actionable recommendations 130A based on the plurality of probability values corresponding to the plurality of predefined medical conditions 306. Further, in some embodiments, a lookup table 506 associates a plurality of probability ranges of the plurality of predefined medical conditions 306 with a plurality of predefined actionable recommendations 530. The computer system 200 identifies a subset of the plurality of probability ranges for a subset of predefined medical conditions 306. Based on the subset of probability ranges for the subset of predefined medical conditions, the lookup table 506 is checked to identify the first set of actionable recommendations 130A from the plurality of predefined actionable recommendations 530.

In some embodiments, the plurality of predefined medical conditions 306 include a first medical condition 306-1 corresponding to a first probability value 502-1 (shown as Disease 1 Data, etc.). The computer system 200 compares the first probability value 502-1 to a first threshold to determine the first set of actionable recommendations 130A. Further, in some embodiments, the plurality of predefined medical conditions 502 includes a second medical condition 502 corresponding to a second probability value 502-2. The computer system 200 compares the second probability value 502-2 to a second threshold to adjust the first set of actionable recommendations 130A determined based on the first probability value 502-1 of the first medical condition 306-1. Additionally, in some embodiments, the first medical condition 306-1 is diabetes, and the second medical condition 306-2 is one or more of hypertension, cancer, arthritis, or asthma.

In some embodiments, the condition vector includes an activity level. Based on the activity level, the computer system 200 adjusts the first set of actionable recommendations 130A determined based on the first probability value 502-1 of the first medical condition 306-1.

In some embodiments, the plurality of predefined medical conditions 306 include one or more of: asthma, heart disease, stroke, diabetes, arthritis, cancer, obesity, Alzheimer's disease, substance abuse, influenza, HIV, Zoonotic disease, tuberculosis, chronic kidney disease, and mental illness.

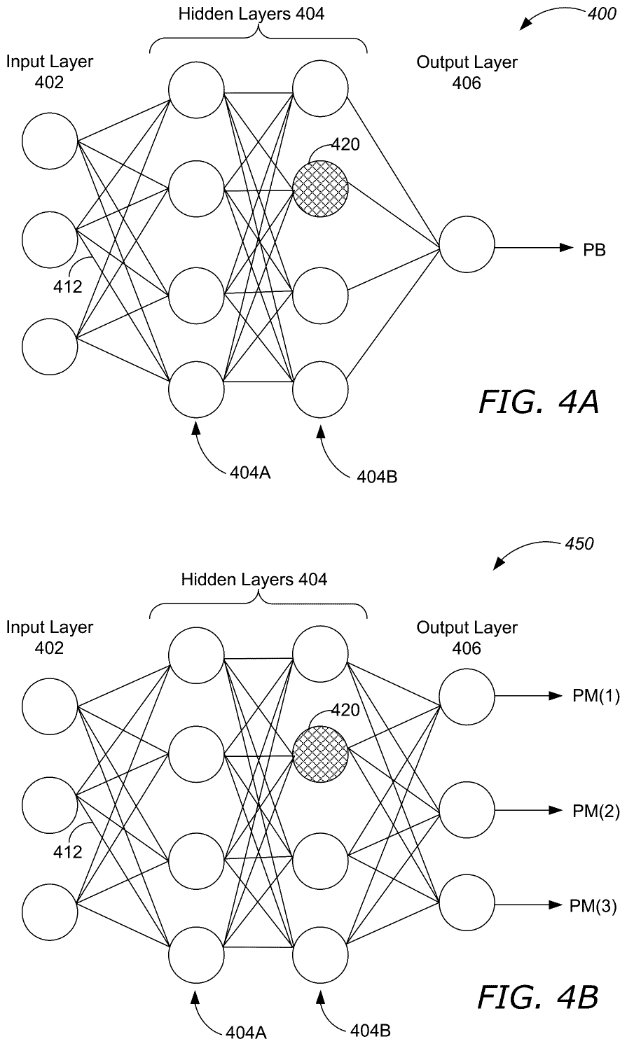

In some embodiments, the computer system 200 applies the classifier models 302 to the digital model 242 to identify the first set of actionable recommendations 130A by generating, by the classifier model 302, an action vector having a plurality of recommendation scores 508 (as depicted below in the discussion of FIG. 4) corresponding to a plurality of predefined actionable recommendations 530, ranking the plurality of recommendation scores 508 corresponding to the plurality of predefined actionable recommendations 530, and selecting the first set of actionable recommendations 130A having the highest recommendation scores 508 in the plurality of predefined actionable recommendations 530.

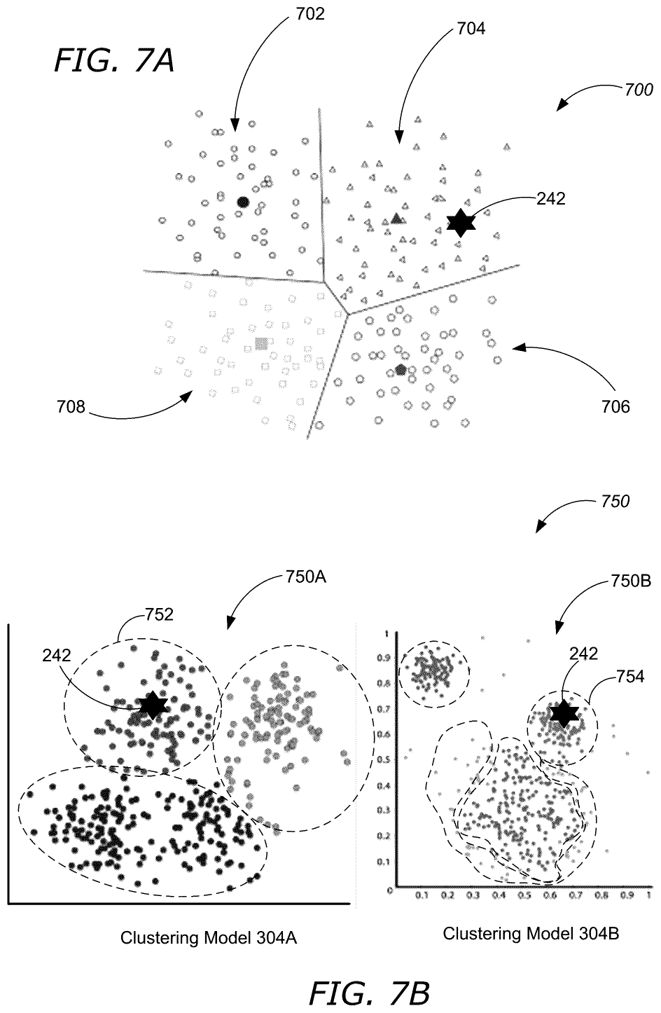

In some embodiments, the one or more clustering models 304 includes a single clustering model 304 corresponds to two or more clusters (e.g., the clusters 702-708 in FIG. 7A) each of which corresponds to one or more cluster recommendations. Further, in some embodiments, each of the two or more clusters corresponds to a respective one of a plurality of predefined medical conditions 306. The plurality of predefined medical conditions 306 include one or more of: asthma, heart disease, stroke, diabetes, arthritis, cancer, obesity, Alzheimer's disease, substance abuse, influenza, HIV, Zoonotic disease, tuberculosis, chronic kidney disease, and mental illness.

In some embodiments, each of the one or more clustering models 304 corresponds to two or more clusters, each of which corresponds to one or more cluster recommendations. The computer system 200 determines the one or more cluster recommendations for each of the respective clusters assigned to the digital model 242 within the one or more clustering models 304 and consolidates the one or more cluster recommendations for each of the respective clusters assigned to the digital model 242 to identify the second set of actionable recommendations 130B. Further, in some embodiments, the computer system 200 resolves conflicts between a first cluster recommendation of a first cluster 752 of a first clustering model 304A and a second cluster recommendation of a second cluster 754 of a second clustering model 304B (as depicted below in the discussion of FIG. 7B).

In some embodiments, each of the one or more clustering models 304 is implemented based on one or more of: a K-means clustering method, a density-based spatial clustering of applications with noise (DBSCAN) clustering method, a Gaussian Mixture Model method, a balance iterative reducing and clustering using Hierarchies (BIRCH), an Affinity Propagation clustering method, a mean-shift clustering method, an ordering points to identify the clustering structure (OPTICS) method, or an agglomerative hierarchy clustering method.

In some embodiments, each of the one or more clustering models 304 is density-based, distribution-based, centroid-based, or hierarchical based. In more embodiments, the computer system 200 trains the classifier models 302 using first user data and corresponding ground truth and trains the one or more clustering models 304 using the first user data without the corresponding ground truth. In further embodiments, each of the first and second sets of actionable recommendations 130A and 130B is one of: a medication with a dosage, an activity with an activity level, a food recipe, or a dietary supplemental with a suggested usage.

Prior to presenting the subset of the first and second sets of actionable recommendations, the computer system 200 identifies any inconsistency or conflict between one of the first set of actionable recommendations 130A and one of the second set of actionable recommendations 130B and resolves the inconsistency or the conflict. In some embodiments, resolving the conflict involves presenting a query and receiving a doctor response to the query, automatically disabling both of the actionable recommendations, or automatically, in accordance with a predefined rule, choosing one of the actionable recommendations.

Aspects of the present disclosure may be embodied as an apparatus, system, method, or computer program product. Accordingly, aspects of the present disclosure may take the form of an entirely hardware embodiment, an entirely software embodiment (including firmware, resident software, micro-code, or the like) or an embodiment combining software and hardware aspects that may all generally be referred to herein as a “function,” “module,” “apparatus,” or “system.” Furthermore, aspects of the present disclosure may take the form of a computer program product embodied in one or more non-transitory computer-readable storage media storing computer-readable and/or executable program code. Many of the functional units described in this specification have been labeled as functions, in order to emphasize their implementation independence more particularly. For example, a function may be implemented as a hardware circuit comprising custom VLSI circuits or gate arrays, off-the-shelf semiconductors such as logic chips, transistors, or other discrete components. A function may also be implemented in programmable hardware devices such as via field programmable gate arrays, programmable array logic, programmable logic devices, or the like.

Functions may also be implemented at least partially in software for execution by various types of processors. An identified function of executable code may, for instance, comprise one or more physical or logical blocks of computer instructions that may, for instance, be organized as an object, procedure, or function. Nevertheless, the executables of an identified function need not be physically located together but may comprise disparate instructions stored in different locations which, when joined logically together, comprise the function and achieve the stated purpose for the function.

Indeed, a function of executable code may include a single instruction, or many instructions, and may even be distributed over several different code segments, among different programs, across several storage devices, or the like. Where a function or portions of a function are implemented in software, the software portions may be stored on one or more computer-readable and/or executable storage media. Any combination of one or more computer-readable storage media may be utilized. A computer-readable storage medium may include, for example, but not limited to, an electronic, magnetic, optical, electromagnetic, infrared, or semiconductor system, apparatus, or device, or any suitable combination of the foregoing, but would not include propagating signals. In the context of this document, a computer readable and/or executable storage medium may be any tangible and/or non-transitory medium that may contain or store a program for use by or in connection with an instruction execution system, apparatus, processor, or device.

Computer program code for carrying out operations for aspects of the present disclosure may be written in any combination of one or more programming languages, including an object-oriented programming language such as Python, Java, Smalltalk, C++, C#, Objective C, or the like, conventional procedural programming languages, such as the “C” programming language, scripting programming languages, and/or other similar programming languages. The program code may execute partly or entirely on one or more of a user's computer and/or on a remote computer or server over a data network or the like.

A component, as used herein, comprises a tangible, physical, non-transitory device. For example, a component may be implemented as a hardware logic circuit comprising custom VLSI circuits, gate arrays, or other integrated circuits; off-the-shelf semiconductors such as logic chips, transistors, or other discrete devices; and/or other mechanical or electrical devices. A component may also be implemented in programmable hardware devices such as field programmable gate arrays, programmable array logic, programmable logic devices, or the like. A component may comprise one or more silicon integrated circuit devices (e.g., chips, die, die planes, packages) or other discrete electrical devices, in electrical communication with one or more other components through electrical lines of a printed circuit board (PCB) or the like. Each of the functions and/or modules described herein, in certain embodiments, may alternatively be embodied by or implemented as a component.

A circuit, as used herein, comprises a set of one or more electrical and/or electronic components providing one or more pathways for electrical current. In certain embodiments, a circuit may include a return pathway for electrical current, so that the circuit is a closed loop. In another embodiment, however, a set of components that does not include a return pathway for electrical current may be referred to as a circuit (e.g., an open loop). For example, an integrated circuit may be referred to as a circuit regardless of whether the integrated circuit is coupled to ground (as a return pathway for electrical current) or not. In various embodiments, a circuit may include a portion of an integrated circuit, an integrated circuit, a set of integrated circuits, a set of non-integrated electrical and/or electrical components with or without integrated circuit devices, or the like. In one embodiment, a circuit may include custom VLSI circuits, gate arrays, logic circuits, or other integrated circuits; off-the-shelf semiconductors such as logic chips, transistors, or other discrete devices; and/or other mechanical or electrical devices. A circuit may also be implemented as a synthesized circuit in a programmable hardware device such as field programmable gate array, programmable array logic, programmable logic device, or the like (e.g., as firmware, a netlist, or the like). A circuit may comprise one or more silicon integrated circuit devices (e.g., chips, die, die planes, packages) or other discrete electrical devices, in electrical communication with one or more other components through electrical lines of a printed circuit board (PCB) or the like. Each of the functions and/or modules described herein, in certain embodiments, may be embodied by or implemented as a circuit.

Reference throughout this specification to “one embodiment,” “an embodiment,” or similar language means that a particular feature, structure, or characteristic described in connection with the embodiment is included in at least one embodiment of the present disclosure. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to”, unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Further, as used herein, reference to reading, writing, storing, buffering, and/or transferring data can include the entirety of the data, a portion of the data, a set of the data, and/or a subset of the data. Likewise, reference to reading, writing, storing, buffering, and/or transferring non-host data can include the entirety of the non-host data, a portion of the non-host data, a set of the non-host data, and/or a subset of the non-host data.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps, or acts are in some way inherently mutually exclusive.

Aspects of the present disclosure are described below with reference to schematic flowchart diagrams and/or schematic block diagrams of methods, apparatuses, systems, and computer program products according to embodiments of the disclosure. It will be understood that each block of the schematic flowchart diagrams and/or schematic block diagrams, and combinations of blocks in the schematic flowchart diagrams and/or schematic block diagrams, can be implemented by computer program instructions. These computer program instructions may be provided to a processor of a computer or other programmable data processing apparatus to produce a machine, such that the instructions, which execute via the processor or other programmable data processing apparatus, create means for implementing the functions and/or acts specified in the schematic flowchart diagrams and/or schematic block diagrams block or blocks.

It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated figures. Although various arrow types and line types may be employed in the flowchart and/or block diagrams, they are understood not to limit the scope of the corresponding embodiments. For instance, an arrow may indicate a waiting or monitoring period of unspecified duration between enumerated steps of the depicted embodiment.

In the following detailed description, reference is made to the accompanying drawings, which form a part thereof. The foregoing summary is illustrative only and is not intended to be in any way limiting. In addition to the illustrative aspects, embodiments, and features described above, further aspects, embodiments, and features will become apparent by reference to the drawings and the following detailed description. The description of elements in each figure may refer to elements of proceeding figures. Like numbers may refer to like elements in the figures, including alternate embodiments of like elements.

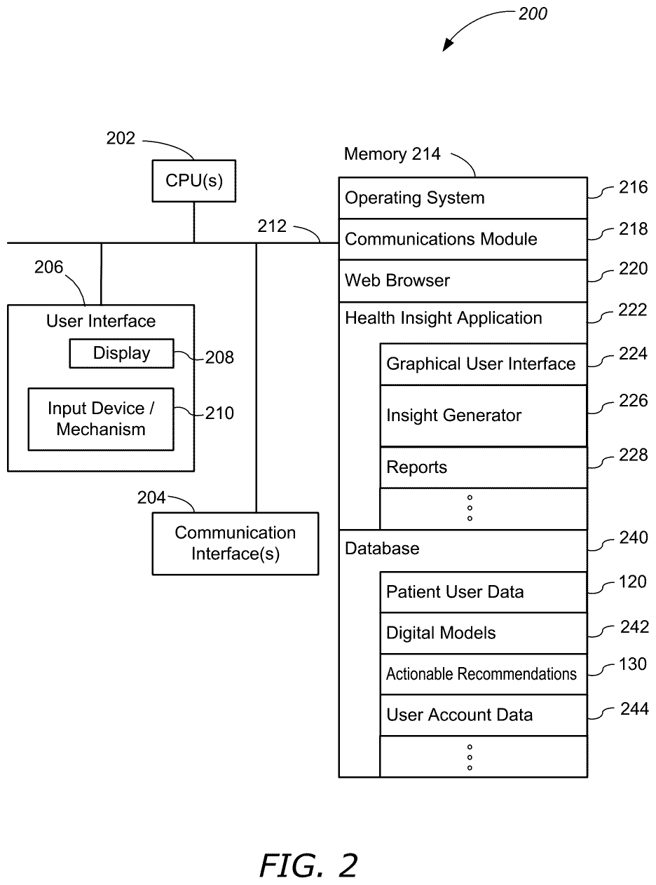

FIG. 1 is a block diagram illustrating an example network architecture that enables a multi-user data management system 100, in accordance with some embodiments. The multi-user data management system 100 includes one or more servers 102 (e.g., the servers 102A and 102B), a plurality of electronic devices 104 (e.g., devices 104A and 104B) associated with medical institutions 106 (e.g., hospitals 106A, doctor offices 106B, and labs 106C), and a plurality of client devices 108 (e.g., user devices 108A, 108B, and 108C) associated with individual patient users 110. The one or more servers 102 hosts a multi-user medical data application (e.g., a health insight application 222 in FIG. 2) configured to collect demographic and user-specified medical characteristics 120A, historical medical data 120B, activity data 120C, and biomarker data 120D for each of a plurality of patient users 110 from the electronic devices 104 and client devices 108 and generate respective actionable recommendations 130 customized for each patient user 110. The electronic devices 104 associated with the patient users 110 may be, for example, desktop computers, tablet computers, medical equipment, or intelligent, multi-sensing, network-connected sensor devices used in the medical institutions 106. The electronic devices 104 collect, pre-process, and store demographic and medical data for each patient user 110 associated with the patient user's visits to the medical institutions 106. The respective actionable recommendations 130 are presented adaptively on a respective client device 108 of each patient user 110. The client devices 108 of the patient users 110 may be, for example, desktop computers 108A, tablet computers, mobile phones 108B, or intelligent, multi-sensing, network-connected wearable devices 108C (e.g., an Apple Watch or a Fitbit wristband). The respective actionable recommendations 130 are presented adaptively on a respective client device 108 of each patient user 110. In some embodiments, the multi-user data management system 100 further includes storage 112 for storing data related to the servers 102, electronic devices 104, client devices 108, and applications executed thereon.

The server(s) 102, electronic devices 104, client devices 108, and storage 112 are communicatively coupled to each other via one or more communication networks 114, which are the medium used to provide communications links between these devices and computers connected together within the multi-user data management system 100. The one or more communication networks 114 may include connections, such as wire, wireless communication links, or fiber optic cables. Examples of the one or more communication networks 114 include local area networks (LAN), wide area networks (WAN) such as the Internet, or a combination thereof. The one or more communication networks 114 are implemented using any known network protocol, including various wired or wireless protocols, such as Ethernet, Universal Serial Bus (USB), FIREWIRE, Long Term Evolution (LTE), Global System for Mobile Communications (GSM), Enhanced Data GSM Environment (EDGE), code division multiple access (CDMA), time division multiple access (TDMA), Bluetooth, Wi-Fi, voice over Internet Protocol (VOIP), Wi-MAX, or any other suitable communication protocol. A connection to the one or more communication networks 114 may be established either directly (e.g., using 3G/4G connectivity to a wireless carrier), or through a network interface 116 (e.g., a router, switch, gateway, hub, or an intelligent, dedicated whole-home control node), or through any combination thereof. As such, in some embodiments, the one or more communication networks 114 can represent the Internet of a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other electronic systems that route data and messages.

In some embodiments, the one or more servers 102 include a recommendation server 102A and one or more third-party servers 102B. Each third-party server 102B is communicatively coupled, and configured to provide data management service, to a subset of the electronic devices 104 and the client devices 108. For example, a third-party server 102B is configured to execute a medical data application for managing medical data (e.g., visit summaries, medication dosage, and/or blood test results) for patient users 110 that visit different sites of a large hospital. In another example, another third-party server 102B is configured to execute a mobile fitness application for tracking exercises data for patient users 110 via their associated mobile phones 108B or wearable devices 108C. In some embodiments, the recommendation server 102A is coupled to the one or more third-party servers 102 and configured to obtain the demographic, medical, and activity data 120 of each of a plurality of patient users 110 from the one or more third-party servers 102B. Alternatively, in some embodiments, the recommendation server 102A is configured to obtain a subset of the demographic, medical, and activity data 120 of each of a plurality of patient users 110 from the electronic devices 104 and client devices 108.

The demographic, medical, and activity data 120 (e.g., the data 120-120D) for each patient user 110 collected by the recommendation server 102A corresponds to a plurality of features that forms a digital model of the respective patient user 110. In some embodiments, the demographic, medical, and activity data 120 includes user-specified medical characteristics 120A, historical medical data 120B, activity data 120C, biomarker data 120D, and changes over time in the user-specified medical characteristics 120A, the historical medical data 120B, the activity data 120C, and the biomarker data 120D of the user 110. The user-specified medical characteristics 120A are directly provided by the user 110 (e.g., input via a client device 108 associated with the user 110). The historical medical data 120B of the user 110 is stored in, and retrieved from, a database of medical records of a medical institution 106. The activity data 120C is collected from one or more wearable devices 108C worn by the user 110, and, in some embodiments, retrieved using an Application Programming Interface (API). The biomarker data 120D is measured (e.g., in a laboratory 106C) according to one or more laboratory tests of bodily fluids extracted from the user 110. Further, in some embodiments, the digital model has a predefined data structure in which the plurality of features is organized to be used as inputs to machine learning models, which are implemented by the recommendation server 102.

In some embodiments, the one or more servers 102 host a medical data application (e.g., the health insight application 222 in FIG. 2) for a large number of patient users 110 and act as a digital hub that integrates a subset or all of: user-specified medical characteristics 120A, historical medical data 120B, activity data 120C, biomarker data 120D, API data, purchase history, self-reported data, and data stored in an electronic knowledge database of health-related data. As the digital hub, the servers 102 monitor, track, and display each patient user's health data, physical activity data, internal inputs and outputs against the user's individual historical data and the data from the electronic knowledge database of health-related data. The recommendation server 102A also provide insights, suggestions, recommendations 130, support, motivation, and direction to advice and care, thereby fostering user behaviors associated with immediate and long-term health.

In some embodiments, a digital model or a digital twin is a digital representation of a patient user 110, contextualized in a digital version of the multi-user data management system 100. The terms “digital model” and “digital twin” are used interchangeably. In some embodiments, the digital model of the patient user 110 includes one or more digital twins that are constructed based on the biomarker data 108D, self-report behavioral data of the user-specified medical characteristics 120A, the historical medical data 120B, and the activity data 120C. The server 102A compares a digital twin of the patient user 110 with other digital twins of other patient users 110 using machine learning (embeddings) to identify similar digital twins to provide lifestyle and health suggestions.

In some embodiments, “medical data” broadly includes lab test results including biomarker data 108D, biometrics, medical records including the historical medical data 120B, supplement order history, prescription history, self-reported health and demographic data (e.g., in user-specified medical characteristics 120A), medical history, health data measured by the wearable device 108C, the activity data 120C, and other data that is relevant to a person's health and activities. Based on the medical data, the server(s) 102 are configured to generate one or more actionable recommendations 130. Such actionable recommendations 130 are applied to deliver products and services including one or more of: consultation to healthcare providers, nutritionists, dieticians, coaches, and fitness instructors; chatbots that collect additional information and make algorithmic suggestions; customized supplement formulas and packs; optional autoshipping; off-the-shelf supplement recommendations 130; food and drink delivery planning; food and drink guidance and planning; fitness recommendations 130; fitness plans; behavioral guidance and planning; pharmaceutical prescriptions; peptide, exosome, and stem cell therapies and treatments; alternative therapeutics; referrals to specialists; additional tests; wearable and hardware recommendations; doctor referral; hepatologist referral; hardware delivery; recommendation for liquid biopsy: APIs for companies to integrate with medical data; community support groups (e.g., people with similar thyroid conditions); liquid biopsy delivery based on self-reported and biomarker data; emergency care based on medical data (e.g., receiving a house call with an IV if blood test results reveal that the user is dangerously dehydrated or low on potassium); and tests suggested and offered based on supplement purchasing history.

In some embodiments, the server(s) 102 collect lab test data including the biomarker data 120D with wearable data measured by the client device 108, and provide recommendations 130 including safety alerts. In some embodiments, the server(s) 102 collect lab test data including the biomarker data 120D with wearable data measured by the client device 108, and provide recommendations 130 and plans customized for each patient user 110. For example, a person's wearable device 108C (e.g., an Oura ring) is used to present a recommendation 130 to go to sleep early, and the recommendation 130 is generated in accordance with detection of a waning testosterone level, which is caused by sleep deprivation.

In some embodiments, the server(s) 102 provide recommendations 130 including reminders for behavioral modification based on the medical data including data 120A-120D and associated changes over time. For example, in accordance with a determination that a user's blood sugar level is above a blood sugar threshold, the server(s) 102 provide a reminder in real time to recommend having a protein-rich breakfast. In some embodiments, the server(s) 102 automatically provide recommendations 130 including additional tests, supplements, fitness, and health plans based on the medical data. In some embodiments, the server(s) 102 integrate medical data with food and behavior logging, and provide recommendations 130. In some situations, the recommendations 130 includes a yes/no response to a user inquiry (e.g., asking whether a sugar-rich dissert is acceptable given a user's current glucose level). In some embodiments, a toilet sensor is installed to collect data of a patient user's urine or data of a patient user's toilet behavior, and the collected data is integrated with other medical data (e.g., the data 120A-120D and associated changes over time) to provide a recommendation 130 to the patient user 110.

In some embodiments, the server(s) 102 integrate medical data (e.g., data 120A-120D and associated changes over time) of family members, identify overlaps at a period of time, and determine trends (e.g., glucose increase) among the family members. Stated another way, the medical data of the family members are clustered to determine characteristics of their associated cluster.

In some embodiments, the server(s) 102 share demographic, medical, and activity data 120 of patient users 110 to achieve some purposes. For example, the data of the patient users 110 is shared to qualify for a product or service (e.g., a provider fitness plan, a gov't benefit, insurance, travel, food programs, or home care). In some embodiments, the data of the patient users 110 is shared between both medical and non-medical services. In some embodiments, the data of the patient users 110 is shared to qualify for paid and unpaid research studies (e.g., to identify similar patient users 110). In some embodiments, the data of the patient users 110 is shared to inform a third party (a chef, a food company, a health coach, or a fitness provider), so that third parties can better tailor their services. In some embodiments, the data of the patient users 110 is shared to apply for a job or role. In some embodiments, the data of the patient users 110 is shared for dating or communities (e.g., an STI profile or overall health). In some embodiments, the data of the patient users 110 is shared to qualify for support communities (e.g., people with thyroid disorders). In some embodiments, the data of the patient users 110 is shared with family and friends in case of emergency. In some embodiments, the data of the patient users 110 is shared with one or more third parties (e.g., providers, coaches, family, friends, restaurants, gyms, or insurance providers). In some embodiments, the data of the patient users 110 is shared to allow a patient user 110 to opt-in to becoming a data donor (e.g., end of life). In these embodiments, the server(s) have to authenticate an associated computer system or server 102 before sharing.

In some embodiments, the data management system 100 delivers unique content (e.g., actionable recommendations 130) customized based on each person's demographic, medical, and/or activity data 120. In some embodiments, the data management system 100 combines content items based on the demographic, medical, and/or activity data 120 into a custom piece of content.

In some embodiments, the data management system 100 uses demographics to inform medical and non-medical recommendations 130. For example, demographic data can be used as background information, such as a self-reported Jewish woman testing for the BRCA gene to ensure breast cancer predisposition is not present. In another example, geographic data is relevant when there are known risks associated with particular geographic locations, such as heavy metals like cadmium found nearby. The data management system 100 recommends a cadmium lab test and offers a plan and supplements to chelate cadmium from the body. In some instances, a geographic correlation can be identified even without knowing a specific cause. For example, a person 110 is living in an area with a population known for diabetes, and the data management system 100 recommends glucose testing.

Some embodiments use computer vision. In some embodiments, the data management system 100 requests a selfie image (e.g., captured in real time using a mobile phone), integrates the selfie image with medical data, and tracks changes over time. For example, the data management system 100 analyzes skin conditions of a patient user 110. In some embodiments, object character recognition (OCR) techniques are applied to recognize food labels and/or menus, and food intake information is compared with the user's medical data to offer suggestions. In some situations, the data management system 100 points out that consuming this food is beneficial or harmful.

In some embodiments, the data management system 100 is configured to provide automated underwriting of health and life insurance based on medical data. In some embodiments, the data management system 100 provides qualified offers. In some embodiments, the data management system 100 uses identified data to inform patient users 110 of insurance offers.

Some embodiments use the health data for the benefit of a large population of a community. For example, the data management system 100 identifies a test population (e.g., within a company, within a county, or within a city) to inform stakeholders who provide services and products of health data. In some embodiments, the data management system 100 is configured to identify health issues in an aggregate group, rather than in individual patient users 110. For example, the data management system 100 is configured to inform a corporation that its employees need a new benefit for thyroid health given many instances of thyroid disorders spotted by lab test results, utilize county health data to regulate food services, uncover genetic and social-determinant-of-health with robust testing, and display a macro dashboard to visualize a population's health.

Some embodiments enable searches based on medical data as a filter. The data management system 100 is configured to search for target medical information in a person's medical data and select and sort the search results accordingly (e.g., for content and offerings). In an example regarding a search at a supplement store, the data system 100 determines that a patient user 100 has a thyroid condition and provides recommendations 130 based on the thyroid condition. In another example, a content site delivers content based only on the medical data. In another example, the data management system 100 is configured to generate recommendations 130 for doctors that are appropriate for each user based on the medical data of the patient user (e.g., apply the medical data of the patient user as a filter). In some embodiments, the data management system 100 is configured to filter medical data to recommend associated medical professionals. Further, in some situations, the data management system 100 reaches out to the recommended medical professionals anonymously (e.g., using an API). In some embodiments, the data management system 100 is configured to use searching to help identify malpractice. For example, the data management system 100 identifies anomalous healthcare prescriptions, suggestions, and/or errors that is an outlier among a large set of medical data or compared with known normal medical data.

In some embodiments, the data management system 100 applies gamification of biomarker metrics. For example, the data management system 100 sets up biomarkers goals (e.g., a reachable target) and enables a patient user 110 to compare and compete with other patient users 110. Further, in some embodiments, rewards and badges are provided to encourage improvement or adherence, thereby effectively improving health conditions of the patient user 110.

In various embodiments of this application, demographic, medical, and activity data 120 of a plurality of patient users 110 is provided to the server(s) 102 and processed by machine learning. Such data is provided by the electronic devices 104 and client devices 108 associated with the plurality of patient users 110, and includes one or more of: lab results including biomarker data 120D, historical medical data 120B, API data, and user-specified medical characteristics 120A. Based on such data, the server(s) 102 create a digital twin (also called a digital model), compares the digital twin to other digital twins in the system to inform a deeper and more comprehensive set of results, and utilizes the newly created digital twin as a data set, for comparison and analysis with other members' digital twins. In some embodiments, one or more classifier models and one or more clustering models are applied to identify actionable recommendations 130 to a patient user 110 based on his or her digital twin. In this application, a medical doctor is enabled to rely on computational resources of the server(s) 102 to compile a large number of patient users' data from different touchpoints and evaluate the data with instant and mechanical certainty against an ever-expanding set of identical (albeit variable) data points. A medical doctor is therefore enabled to perform continuous and real-time data analysis, particularly analyze data across a large number of patient users 110 visiting different medical institutions 106 and in different locations. By these means, as each individual patient user's condition is analyzed in the context of the large number of other patient users 110, the actionable recommendations 130 are made in light of emerging health trends (e.g., evolving repercussion of long COVID) and statistically significant relationships among variables, which were not considered previously due to computational limitations of human-driven research.

FIG. 2 is a block diagram illustrating a representative computer system 200, in accordance with some embodiments. The computer system 200 is configured to obtain demographic, medical, and activity data 120 for a plurality of patient users 110, apply machine learning to process the demographic, medical, and activity data 120, and generate actionable recommendations 130 to be presented to the patient users 110. In some embodiments, the computer system 200 includes one or more servers 102 that execute a health insight application 222 for which a graphical user interface 224 is enabled to display the actionable recommendations 130 on each patient user's client device 108. Various examples of the computer system 200 include a desktop computer, a laptop computer, a tablet computer, a smart phone, and other computing devices that have a processor capable of hosting the health insight application 222 for the plurality of patient users 110. The computer system 200 typically includes one or more processing units/cores (CPUs) 202 for executing modules, programs, and/or instructions stored in the memory 214 and thereby performing processing operations; one or more network or other communications interfaces 204; memory 214; and one or more communication buses 212 for interconnecting these components. The communication buses 212 may include circuitry that interconnects and controls communications between system components.

In some embodiments, the computer system 200 includes a user interface device 206 comprising a display device 208 and one or more input devices or mechanisms 210. In some embodiments, the input device/mechanism includes a keyboard. In some embodiments, the input device/mechanism includes a “soft” keyboard, which is displayed as needed on the display device 208, enabling a user to “press keys” that appear on the display 208. In some embodiments, the display 208 and input device/mechanism 210 comprise a touch screen display (also called a touch sensitive display).

In some embodiments, the memory 214 includes high-speed random-access memory, such as DRAM, SRAM, DDR RAM or other random-access solid-state memory devices. In some embodiments, the memory 214 includes non-volatile memory, such as one or more magnetic disk storage devices, optical disk storage devices, flash memory devices, or other non-volatile solid state storage devices. In some embodiments, the memory 214 includes one or more storage devices remotely located from the CPU(s) 202. The memory 214, or alternatively the non-volatile memory device(s) within the memory 214, comprises a non-transitory computer readable storage medium. In some embodiments, the memory 214, or the computer readable storage medium of the memory 214, stores the following programs, modules, and data structures, or a subset thereof:
an operating system 216, which includes procedures for handling various basic system services and for performing hardware dependent tasks;
a communications module 218, which is used for connecting the computer system 200 to other computers and devices via the one or more communication network interfaces 204 (wired or wireless) and one or more communication networks, such as the Internet, other wide area networks, local area networks, metropolitan area networks, and so on;
a health insight application 222, which enables display of a graphical user interface 224 on a patient user's client device 108, where the health insight application 222 includes an insight generator 226 for applying machine learning to process demographic, medical, and activity data 120 of a patient user 110 and determine actionable recommendations 130; and
zero or more databases 240, which store demographic, medical, and activity data 120 (collectively called patient user data 120) of a plurality of patient users 110, digital models 242, actionable recommendations 130, and/or user account data 244. In some embodiments, the database is stored as spreadsheet files, CSV files, XML files, or flat files, or stored in a relational database.

In some embodiments, the health insight application 222 provides reports 228 for the patient user 110. In some embodiments, the health insight application 222 executes within the web browser 220 or another application using web pages provided by a third-party web server 102B. In some embodiments, the health insight application 222 executes as a standalone downloaded application.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 214 stores a subset of the modules and data structures identified above. Furthermore, the memory 214 may store additional modules or data structures not described above.

Although FIG. 2 shows a computer system 200, FIG. 2 is intended more as a functional description of the various features that may be present rather than as a structural schematic of the embodiments described herein. In practice, and as recognized by those of ordinary skill in the art, items shown separately could be combined and some items could be separated.

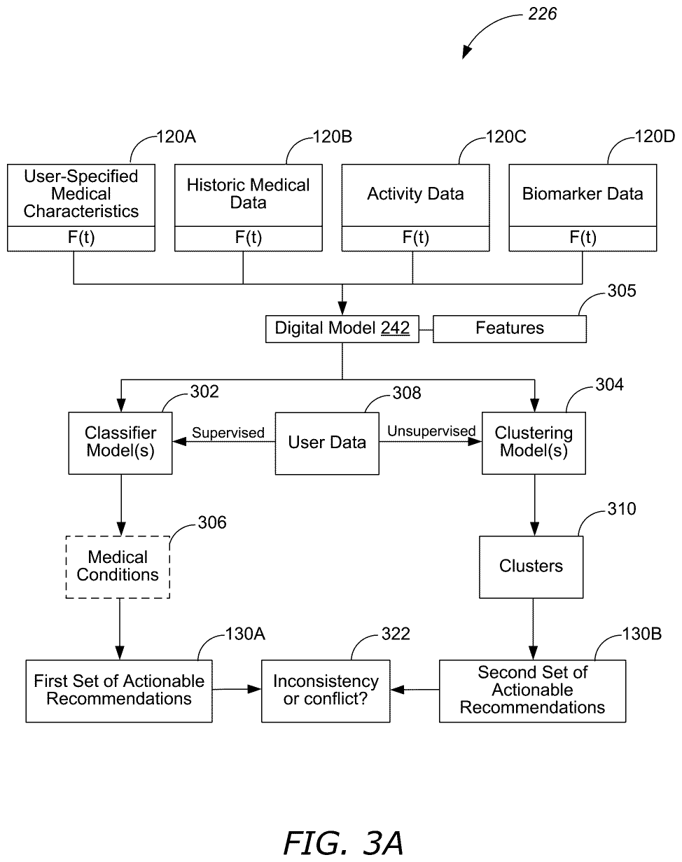

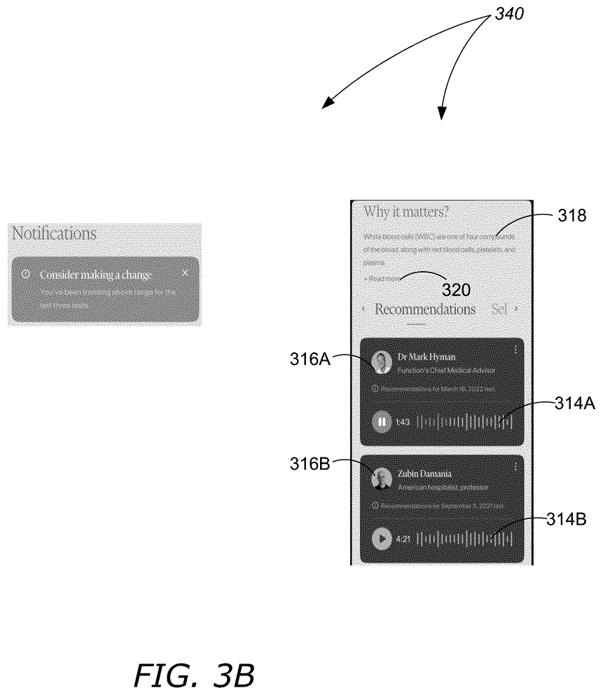

FIG. 3A is a block diagram of an example insight generator 226 of a data processing system 100 that generates actionable recommendations 130, in accordance with some embodiments, and FIG. 3B illustrates user interfaces 340 displaying actionable recommendations 130, in accordance with some embodiments. In some embodiments, the insight generator 226 is implemented on a computer system 200 (e.g., a recommendation server 102A), and includes one or more classifier models 302 and one or more clustering models 304. Specifically, the insight generator 226 obtains demographic, medical, and activity data 120 of a patient user 110, and constructs a digital model 242 (also called a digital twin) of the patient user 110 including a plurality of features 305 to represent the demographic, medical, and activity data 120 of the patient user 110. The computer system 200 applies the classifier models 302 to the digital model 242 to determine a first set of actionable recommendations 130A (e.g., by way of identifying one or more medical conditions 306 of the patient user 110). The computer system 200 identifies the one or more clustering models 304 generated from user data 308 for a plurality of users 110 in a vector space having dimensions correspond to the plurality of features 305. The digital model 242 including the plurality of features 305 is assigned to a respective cluster within each of the one or more clustering models 304 and identifies a second set of actionable recommendations 130B according to respective clusters 310 assigned to the digital model 242 within the one or more clustering models 304. At least a subset of the first and second sets of actionable recommendations 130A and 130B is presented to the patient user 110 on a user interface 340.

In some embodiments, the demographic, medical, and activity data 120 applied to generate the digital model 242 of the user 110 includes a subset of: user-specified medical characteristics 120A, historical medical data 120B, activity data 120C, and biomarker data 120D of the user 110. Examples of the user-specified medical characteristics 120A include, but are not limited to, age, gender, height, weight, medication, medical symptoms, mental health conditions, and family medical history. The historical medical data 120B was recorded during visits to hospitals 106A and doctor's offices 106B and is retrieved from databases of medical records of medical institutions 106. In some embodiments, the historical medical data 120B include medical images obtained via an ultrasound, X-ray, Magnetic Resonance Imaging (MRI), or Positron Emission Tomography (PET) scan. The activity data 120C is measured by one or more wearable devices 108C (e.g., an Apple Watch, a Fitbit wristband, or an Oura ring) worn by the user 110. The biomarker data 120D is measured (e.g., in a lab 106C) by one or more laboratory tests of the patient user's bodily fluids. Further, in some embodiments, the demographic, medical, and activity data 120 includes changes over time (e.g., F(t)) in the user-specified medical characteristics 120A, the historical medical data 120B, the activity data 120C, and the biomarker data 120D.

In some embodiments, the plurality of features 305 of the digital model 242 is generated by a feature extraction model (e.g., including a series of convolutional and pooling layer pairs) that processes the demographic, medical, and activity data 120 of the patient user 110 to generate the plurality of features 305 of the digital model 242 of the patient user 110. The digital model 242 has a predefined data structure in which the plurality of features 305 is organized to be used as inputs to the classifier models 302 and the one or more clustering models 304. In an example, the plurality of features 305 is organized in a feature vector including an ordered sequence of features.

In some embodiments, the user data 308 used to generate the one or more clustering models 304 is distinct from the demographic, medical, and activity data 120, and includes training data that is applied to train the one or more clustering model 304 in an unsupervised manner. Further, in some embodiments, the user data 308 includes training data that is applied to train the classifier models 302 in a supervised manner. The user data 308 therefore includes ground truth corresponding to the training data.

In some embodiments, a medical data application (e.g., a health insight application 222) is executed by the server(s) 102 to host a multi-user data processing platform for a large number of patient users 110. Referring to FIG. 3B, a client-side medical data application is executed to display the user interface 340 on a client device 108 associated with a patient user 110. For example, each of the first and second sets of actionable recommendations 130A and 130B is one of: a medication with a dosage, an activity with an activity level, a food recipe, or a dietary supplemental with a suggested usage. Referring to FIG. 3B, an actionable recommendation 130 (e.g., “Consider making a change”) is displayed with an explanation (e.g., “You've been trending above range for the last three tests.”). In some situations, the subset of actionable recommendation 130 presented to the patient user 110 includes one or more audio clips (e.g., the clips 314A and 314B) provided by one or more different medical professionals (e.g., the professionals 316A and 316B). In some embodiments, the subset of actionable recommendation 130 is presented with one or more general medical tips 318 (e.g., “White blood cells (WBC) are one of four compounds of the blood, along with red blood cells, platelets, and plasma.”). In some embodiments, the user interface 340 includes an actionable affordance 320 (e.g., “+Read more”) configured to provide additional information in response to a user selection of the actionable affordance.

In some embodiments, prior to presenting the subset of the first and second sets of actionable recommendations 130A and 130B, the computer system 200 identifies an inconsistency or conflict 322 between an actionable recommendation in the first set 130A and an actionable recommendation in the second set 130B. The computer system 200 resolves the inconsistency or conflict 322. In some embodiments, the computer system 200 sends a query to a doctor, and receives a doctor response to the query. Alternatively, in some embodiments, the computer system 200 automatically disables both of the actionable recommendations that are in conflict. Alternatively, and additionally, in some embodiments, in accordance with a predefined rule, the computer system 200 automatically chooses one of the actionable recommendations that is in conflict.

In some embodiments, demographic, medical, and activity data 120 of a patient user 110 is encoded over time. In some embodiments, successive readings of one of the demographic, medical, and activity data 120 (e.g., steps walked) is compressed using averages or weighted averages of the readings. The server(s) 102 construct the digital model 242 of the patient user 110 based on the readings directly or based on the averages or weighted averages of the readings. In some embodiments, as demographic, medical, and activity data 120 ages, it is stored (e.g., in the storage 112) in a compressed format. In an example, daily weights in each week are averaged and stored as a weekly weight for the weights that are more than three months ago. In another example, a first reading is measured prior to a second reading, and weighted with a smaller factor than the second reading. In some embodiments, readings of one of the demographic, medical, and activity data 120 are associated with a trend line or curve, and represented with parameters of the trend line or curve (e.g., slope). Some embodiments extrapolate based on the known demographic, medical, or activity data 120, and use other medical data to modify a straight mathematical extrapolation. Some embodiments combine multiple techniques and/or use different techniques for different features 305. As the server(s) 102 construct the digital model 242 of the patient user 110, the digital model 242 includes one or more recent readings of a biomarker and the parameters of the trend line or curve associated with the biomarker.

Medical error is one of the leading causes of death in the United States. Disclosed embodiments help members, and their doctors alike ensure that no critical biomarker result is overlooked, and that red flags and actionable recommendations are promptly and effectively communicated to the user. Moreover, disclosed embodiments help the world understand these very rich data sets in new and important ways. By integrating all of this data and running it through a data processing system 100, which is based on artificial intelligence (e.g., machine learning), disclosed embodiments make connections that enlighten people's medical decisions and health-oriented behaviors.

FIG. 4A is a schematic diagram of a single-class classifier 400 applied for binary classification, in accordance with some embodiments, and FIG. 4B is a schematic diagram of a multi-class classifier 450 applied for multi-class classification, in accordance with some embodiments. Referring to FIG. 4A, the single-class classifier 400 is configured to receive a plurality of features 305 of a digital model 242 of a patient user 110 and generates a single output PB. In some embodiments, a value of the single output PB is binary (e.g., equal to “0” or “1”) indicating whether a predefined class is identified. Alternatively, in some embodiments, the value of the single output PB includes a positive number (e.g., in the interval [0, 1] inclusively) representing a probability of the predefined class. Referring to FIG. 4B, the multi-class classifier 450 is configured to receive the plurality of features 305 of the digital model 242 of the patient user 110 and generates a plurality of output elements (e.g., PM(1), PM(2), and PM(3), which are arranged in an output vector PM). Each output element corresponds to a distinct one of the plurality of predefined classes. In some embodiments, a value of each output element is binary (e.g., equal to “0” or “1”) indicating whether a corresponding predefined class is identified. Alternatively, in some embodiments, the value of each output element includes a positive number (e.g., in the range of [0, 1] inclusively) representing a probability of the corresponding predefined class. In some embodiments, the sum of the plurality of output elements is equal to a predefined sum value (e.g., 1).

Like other neural networks, a classifier 400 or 450 includes a collection of nodes 420 that are connected by links 412. Each node 420 receives one or more node inputs and applies a propagation function to generate a node output from the one or more node inputs. As the node output is provided via one or more links 412 to one or more other nodes 420, a weight w associated with each link 412 is applied to the node output. The one or more node inputs are combined based on corresponding weights w according to the propagation function. In an example, the propagation function is a product of a non-linear activation function and a linear weighted combination of the one or more node inputs.

The collection of nodes 420 is organized into one or more layers in the classifier 400 or 450. In some embodiments, the one or more layers include a single layer acting as both an input layer and an output layer. In some embodiments, the one or more layers includes an input layer 402 for receiving inputs, an output layer 406 for providing outputs, and zero or more hidden layers 404 (e.g., hidden layers 404A and 404B) between the input and output layers 402 and 406. A deep neural network has more than one hidden layer 404 between the input and output layers 402 and 406. In some embodiments, each layer is only connected with its immediately preceding and/or immediately following layer. In some embodiments, an input layer 402 or a hidden layer 404 is a fully connected layer because each node 420 in the input layer 402 or output layer 404 is connected to every node 420 in its immediately following layer. In some embodiments, one of the one or more hidden layers 404 includes two or more nodes that are connected to the same node in its immediately following layer for down sampling or pooling the nodes 420 between these two layers. For example, max pooling uses a maximum value of the two or more nodes in the layer 404B for generating the node of the immediately following layer 406 connected to the two or more nodes.

The training process is a process for calibrating all of the weights w for each layer of the learning model using a training data set that is provided in the input layer 402. The training process typically includes two phases, forward propagation and backward propagation, which are repeated multiple times until a predefined convergence condition is satisfied. In the forward propagation, the set of weights for different layers are applied to the input data and intermediate results from the previous layers. In the backward propagation, a margin of error of the output (e.g., a loss function) is measured, and the weights w are adjusted accordingly to decrease the error. In some embodiments, the activation function is linear, rectified linear, sigmoidal, or a hyperbolic tangent. In some embodiments, a network bias term b is added to the sum of the weighted outputs from the previous layer before the activation function is applied at a node. The network bias b provides a perturbation that helps the classifier 400 or 450 avoid over fitting the training data. The result of the training includes the network bias parameter b for each layer.

FIGS. 5A and 5B are block diagrams of two example classifier subsystems 500 and 520 for providing a first set of actionable recommendations 130A, in accordance with some embodiments. FIGS. 5C and 5D are block diagrams of two example classifier models 302 for providing a first set of actionable recommendations 130A, in accordance with some embodiments. Referring to FIGS. 5A and 5B, in some embodiments, a classifier model 302 includes one or both of: a plurality of first sub-classifier models 302-1 to 302-N and an activity classifier model 302A, where N is a positive integer. Each first sub-classifier model (e.g., the models 302-1, 302-2, . . . , 302-N) is configured to determine respective condition data 502 (e.g., the condition data 502-1 to 502-N in FIG. 5A) of each of a plurality of predefined medical conditions 306 or identify recommendation data 508 (e.g., the recommendations 508-1 to 508-N in FIG. 5B) for a plurality of actionable recommendations 530. The activity classifier model 302A (if any) is configured to determine activity data 504 (e.g., an activity style and an activity level) of the user 110. In some embodiments, each of a first subset of the sub-classifier models 302-1 to 302-N and the activity classifier model 302A includes a single-class classifier 400. In some embodiments, each of a second subset of the sub-classifier models 302-1 to 302-N and activity classifier model 302A includes a multi-class classifier 450. Referring to FIGS. 5C and 5D, in some embodiments, each example classifier model 302 includes a single multi-class classifier 450. The classifier model 302 is configured to determine respective condition data 502 (e.g., the condition data 502-1 to 502-N in FIG. 5C) for each of a plurality of predefined medical conditions 306 or identify recommendation data 508 (e.g., the recommendations 508-1 to 508-N in FIG. 5D) of a plurality of actionable recommendations 530.

Referring to FIGS. 5A and 5C, in some embodiments, the plurality of predefined medical conditions 306 include one or more of: asthma, heart disease, stroke, diabetes, arthritis, cancer, obesity, Alzheimer's disease, substance abuse, influenza, HIV, a Zoonotic disease, tuberculosis, a chronic kidney disease, and mental illness. In some embodiments, the condition data 502 in FIGS. 5A and 5C is binary, indicating whether a predefined medical condition 306 is identified or a positive number (e.g., in the range of [0, 1] inclusively) representing a probability or a severity level of the predefined medical condition 306. In some embodiments, each of the first subset of the sub-classifier models 302-1 to 302-N includes a single-class classifier 400, and a value of a single output PB of the classifier 400 (e.g., condition data 502 in FIG. 5A) is binary (e.g., equal to “0” or “1”) indicating whether a predefined medical condition 306 is identified. Alternatively, in some embodiments, the value of the single output PB of the classifier 400 (e.g., condition data 502 in FIG. 5A) includes a positive number representing a probability or a severity level of the predefined medical condition 306.

In some embodiments, the first set of actionable recommendations 130A is generated based on the plurality of predefined medical conditions 306 (e.g., based on a plurality of probability values of the condition data 502). For each medical condition 306, a respective subset of actionable recommendations 510 (e.g., recommendations 510-1, 510-2, . . . , 510-N) is generated regarding the respective medical condition 306. Further, in some embodiments, the respective subset of actionable recommendations 510 is generated regarding the respective medical condition 306 based on the activity data 504 of the patient user 110. The subsets of actionable recommendations 510 are combined to generate the first set of actionable recommendations 130A.

Alternatively, in some embodiments, a lookup table 506 maps a plurality of combinations of medical conditions 306 to a plurality of actionable recommendations. In accordance with the look-up table 506, the condition data 502 determined by the sub-classifier models 302-1 to 302-N is applied to identify the first set of one or more actionable recommendations 130A. In an example, the lookup table 506 associates a plurality of probability ranges of the plurality of predefined medical conditions 306 with a plurality of predefined actionable recommendations 530 of a pool. The computer system 200 identifies a subset of the plurality of probability ranges 502 of a subset of predefined medical conditions 306 based on the condition data 502. Based on the subset of probability ranges 502 of the subset of predefined medical conditions 306, the lookup table 506 is checked to identify the first set of actionable recommendations 130A from the plurality of predefined actionable recommendations 530 of the pool. Specifically, in some embodiments, the plurality of predefined medical conditions 306 include a first medical condition 306-1 corresponding to a first probability value 502-1. The first probability value 502-1 is compared to a first threshold to determine an actionable recommendation 508-1 of the first set of actionable recommendations 130A. Further, in some embodiments, the plurality of predefined medical conditions 306 includes a second medical condition 306-2 corresponding to a second probability value 502-2. The second probability value 502-2 is compared to a second threshold to adjust the actionable recommendation 508-1 determined based on the first probability value 502-1 of the first medical condition 306-1. In some embodiments, the first medical condition 306-1 includes diabetes, and the second medical condition 306-2 includes one or more of hypertension, cancer, arthritis, or asthma. The diabetes may introduce a set of recommendations, a subset of which does not apply and is eliminated due to other medical conditions of the same patient user 110.

Further, referring to FIG. 5C, in some embodiments, a condition vector is generated to have a plurality of probability values 502 corresponding to the plurality of predefined medical conditions 306. Further, in some embodiments, the condition vector includes an activity level of the activity data 504. Based on the activity level, the computer system 200 adjusts the first set of actionable recommendations 130 determined based on the first probability value of the first medical condition 306-1.

Referring to FIGS. 5B and 5D, in some embodiments, the computer system 200 applies the classifier model 302 or the classifier subsystem 520 to the digital model 242 to identify the first set of actionable recommendations 130A by generating, by the classifier model 302, an action vector having recommendation data 508 including a plurality of recommendation scores corresponding to a plurality of predefined actionable recommendations 530 in a pool, ranking the plurality of recommendation scores corresponding to the plurality of predefined actionable recommendations 530 in the pool, and selecting the first set of actionable recommendations 130A having the highest recommendation scores in the plurality of predefined actionable recommendations.

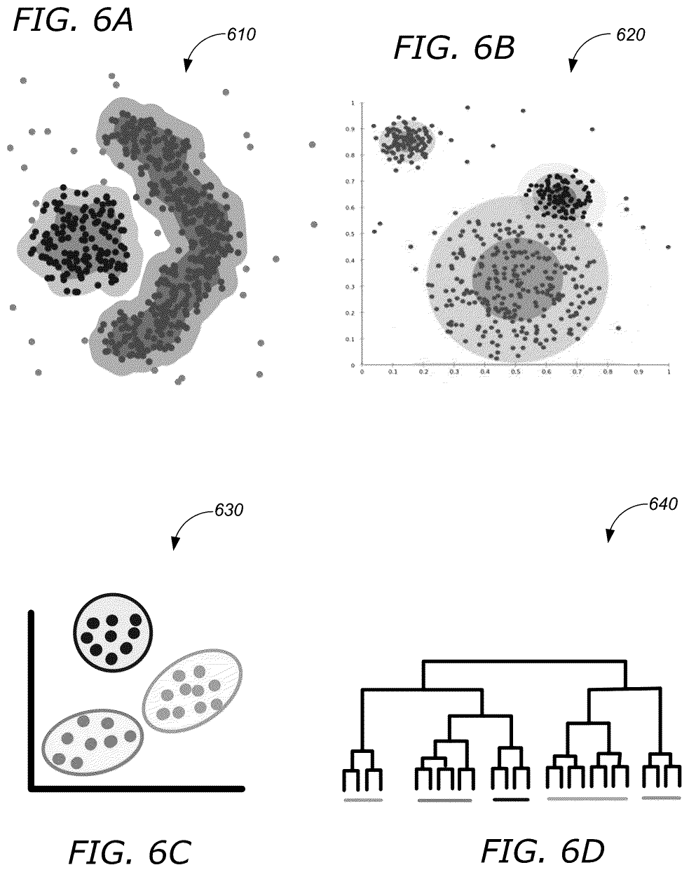

FIGS. 6A-6D illustrate four example clustering models 610, 620, 630, and 640, in accordance with some embodiments. Referring to FIG. 6A, in some embodiments, the clustering model 304 is a density-based clustering model 610. Density-based clustering relies more on density than on distance. Data is clustered by regions of high concentrations of data objects bounded by areas of low concentrations of data objects. The clusters formed are grouped as a maximal set of connected data points. Referring to FIG. 6B, in some embodiments, the clustering model 304 is a distribution-based clustering model 620. Distribution-based clustering creates, and groups data points based on a likelihood of belonging to the same probability distribution (Gaussian, Binomial, etc.) in the data. Referring to FIG. 6C, in some embodiments, the clustering model 304 is a centroid-based clustering model 630 configured to work on closeness of data points to a chosen central value. Datasets are divided into a given number of clusters, and a vector of values references every cluster. The input data variable is compared to the vector value and enters the cluster with minimal difference. The clustering model 304 iteratively measures the distance between the clusters and the characteristic centroids using various distance metrics. These can be, for example, Euclidian distance, Manhattan Distance, and/or Minkowski Distance. Referring to FIG. 6D, in some embodiments, the clustering model 304 is a hierarchical based clustering model 640. Assuming that all data points belong to one large cluster, the computer system 200 divides the data into smaller groups based on termination logic or a point beyond which there will be no further division of data points. In some embodiments, this termination logic is based on the minimum sum of squares of error inside a cluster, or for categorical data. An example metric includes the GINI coefficient inside a cluster. The list of clustering models 610, 620, 630, and 640 is not exhaustive. In some embodiments, the clustering model(s) 304 include a clustering model distinct from any of the clustering models 610, 620, 630, and 640.

In some embodiments, each of the one or more clustering models 304 is implemented based on one or more of: a K-means clustering method, a density-based spatial clustering of applications with noise (DBSCAN) clustering method, a Gaussian Mixture Model method, a balance iterative reducing and clustering using Hierarchies (BIRCH), an Affinity Propagation clustering method, a mean-shift clustering method, an ordering points to identify the clustering structure (OPTICS) method, or an agglomerative hierarchy clustering method.

FIG. 7A illustrates an example single cluster system 700 to which a digital model 242 of a patient user 110 is assigned using associated features 305, in accordance with some embodiments. As explained above, a multi-user data processing system 100 hosts a multi-user medical data application (e.g., the health insight application 222 in FIG. 2) configured to collect demographic, medical, and activity data 120 for each of a plurality of patient users 110 from the electronic devices 104 and client devices 108 and generate respective actionable recommendations 130 customized for each patient user 110. The demographic, medical, and activity data 120 is represented by the digital model 242 including a plurality of features 305. In some embodiments, a feature extraction model includes a series of convolutional and pooling layer pairs, and processes the demographic, medical, and activity data 120 of the patient user 110 to generate the plurality of features 305 of the digital model 242 of a patient user 110. The computer system 200 identifies the clustering model 304 generated from user data 308 for a plurality of users 110 in a vector space having dimensions corresponding to a subset or all of the plurality of features 305. The subset or all of the plurality of features 305 are applied to assign the patient user 110 in the cluster system 700.

The one or more clustering models 304 include a single clustering model associated with the cluster system 700, which includes two or more clusters (e.g., clusters 702, 704, 706, and 708). Each cluster corresponds to one or more respective cluster recommendations. In some embodiments, two clusters have at least one overlapping cluster recommendation. Alternatively, in some embodiments, two clusters have entirely different cluster recommendations. For example, a first cluster 702 corresponds to patient users 110 having light diabetes and severe hypertension, and the one or more cluster recommendations includes practicing yoga. Conversely, a second cluster 704 corresponds to patient users 110 having light diabetes and no hypertension, and the one or more cluster recommendations includes playing tennis or running 5K, which is more intense than practicing yoga.

In some embodiments, the two or more clusters are mutually exclusive. Each of the two or more clusters (e.g., the clusters 702, 704, 706, and 708) corresponds to a respective one of a plurality of predefined medical conditions. A patient user 110 is assigned to a cluster based on the severest medical condition of the user 110. In various embodiments, the plurality of predefined medical conditions can include one or more of: asthma, heart disease, stroke, diabetes, arthritis, cancer, obesity, Alzheimer's disease, substance abuse, influenza, HIV, a Zoonotic disease, tuberculosis, a chronic kidney disease, and mental illness.

FIG. 7B illustrates two example cluster systems 750 to which a digital model 242 of a patient user 110 is assigned using two distinct subsets of features 305, in accordance with some embodiments. Each of the one or more clustering models 304 (e.g., cluster models 304A and 304B) corresponds to a respective cluster system 750 (i.e., cluster systems 750A and 750B) including two or more clusters. Each cluster of a respective cluster system 750 corresponds to one or more cluster recommendations. The computer system 200 determines the one or more cluster recommendations for each of the respective clusters assigned to the digital model 242 within the one or more clustering models. For example, the computer system 200 identifies a first subset of features 305 of the digital model 242, and assigns the patient user 110 to a first cluster 752 in a first cluster system 750A corresponding to a first clustering model 304A. The computer system 200 identifies a second subset of features 305 of the digital model 242, and assigns the patient user 110 to a second cluster 754 in a second cluster system 750B corresponding to a second clustering model 304B. The computer system 200 determines the cluster recommendations for each of the first cluster 752 and the second cluster 754, and consolidates the one or more cluster recommendations for each of the clusters 752 and 754 assigned to the digital model to identify the second set of actionable recommendations 130B.

In some embodiments, a conflict arises between a first cluster recommendation of the first cluster 752 of the first clustering model 304A and a second cluster recommendation of the second cluster 754 of the second clustering model 304B. Further, in some embodiments, one of the first cluster recommendation and the second cluster recommendation is selected as one of the second set of actionable recommendations 130B. Alternatively, in some embodiments, in accordance with detection of the conflict, neither the first cluster recommendation nor the second cluster recommendation is selected as one of the second set of actionable recommendations 130B. Alternatively, and additionally, in some embodiments, in accordance with detection of the conflict, an inquiry is sent to a doctor in charge to determine whether to select the first or the second cluster recommendation. In some embodiments, a set of rules determines which recommendation to use when there is a conflict. The rules can be based on which medical condition has a higher priority or is more severe, a predefined set of priorities, a weighted set of factors that triggered the recommendations, and/or rules generated according to machine learning of recommendation precedence.

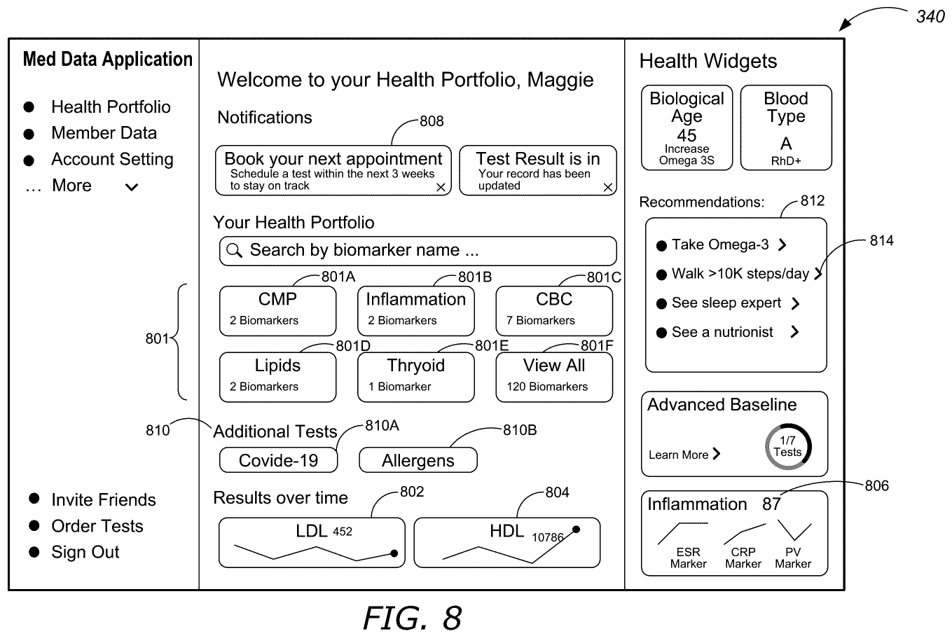

FIG. 8 is another example user interface 340 of a medical data application (e.g., the health insight application 222 in FIG. 2), in accordance with some embodiments. A data processing system 100 hosts the medical data application configured to collect demographic, medical, and activity data 120 for each of a plurality of patient users 110 from the electronic devices 104 and client devices 108 and generate respective actionable recommendations 130 customized for each patient user 110. The medical data application has a plurality of user accounts for the plurality of patient users 110 and a plurality of data accounts for a plurality of medical institutional users 106. Each data account allows a medical institutional user 106 to upload data (e.g., historical medical data 120B and/or biomarker data 120D). Each user account allows a patient user 110 to upload data (e.g., user-specified medical characteristics 120A and/or activity data 120C) and review demographic, medical, and activity data 120, associated analysis results, and actionable recommendations 130A or 130B.

In some embodiments, the user interface 340 includes affordance items 801 (e.g., items 801A-801F) providing access to a health portfolio. In response to a user selection of an affordance item 801A-801E, another user interface or a pop-up window is displayed to present biomarker data 120D associated with the selected affordance item. In response to user selection of an affordance item 801F, another user interface or a pop-up window is displayed to present additional biomarker data 120D or affordance items associated with the additional biomarker data 120D.

The user interface 340 displays medical data including trends of biomarkers over time. Examples trends include, but are not limited to, an LDL trend 802 and an HDL trend 804. Importantly, the interface is designed for patient users 110 who are not medical doctors. The data is displayed in a way that is meaningful to ordinary people. In some embodiments not shown, the dashboard provides data for two or more individuals (e.g., multiple members of a family). In some embodiments, the displayed data illustrates abstract quantifications of medical data trends. Some embodiments also provide combined or aggregated scores, such as an inflammation score 806 based on three biomarkers. Some embodiments also display estimated life expectancy based on the medical data and profile data. In conjunction with life expectancy estimates, some embodiments identify specific factors that a user can address to change the estimated life expectancy. Some embodiments show population health data to stakeholders (e.g., macro views of health).

Some embodiments give automatic suggestions for health providers. This includes automatic insights for coaches and providers, which they can provide to their clients. The data processing system 100 does the work to FIG. out what may be needed. Each provider or coach is empowered to filter the actionable recommendations 130 the system communicates, delivering only what they think is appropriate for the patient users 110. Some embodiments include a dashboard for patient users 110 to interact with. Both patients/clients and providers are able to customize, accept, and reject plans or recommendations based on the relevant demographic, medical, and activity data 120.

In some embodiments, a notification 808 is displayed on the user interface 340 to present an actionable recommendation 130 (e.g., “Book your next appoint”). In some embodiments, additional tests 810 are displayed on the user interface 340 to present an actionable recommendation 130 (e.g., “COVID-19” 810A and “Allergens” 810B), indicating that the patient user 110 should take these tests as recommended. In some embodiments, a recommendation panel 812 is displayed on the user interface 340 and includes a summary of actionable recommendations 130. Each actionable recommendation 130 is displayed with an arrow icon 814. In response to a user selection of the arrow icon 814, another user interface or a pop-up window is displayed to present details of the associated actionable recommendation 130.

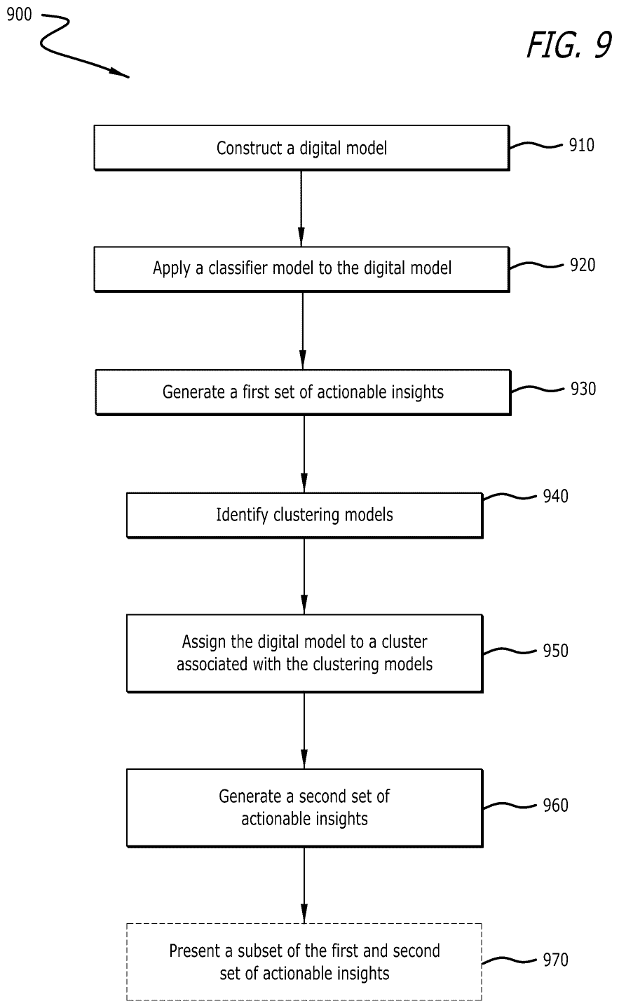

FIG. 9 is a flowchart of a process 900 of managing user data in accordance with some embodiments. In many embodiments, the process 900 can construct a digital model (block 910). As described above, the digital model can include a plurality of data associated with a user that can be processed to generate a plurality of actionable insights. The digital model can be formatted such that it can be utilized as an input in one or more machine learning processes. Those skilled in the art will recognize that the specific format of a digital model can be matched, such as in data format, dimensionality, and/or vector shape.

In a number of embodiments, the process 900 can apply a classifier model to the digital model (block 920). Classifier models may include a number of various types of models, as described above. In some embodiments, the classifier models can be split up into specific or sub-classifier models. These classifiers may also be configured to account for one or more medical conditions that may be associated with a user.

In additional embodiments, the process 900 can generate a first set of actionable insights (block 930). As discussed above, the actionable insights can include a variety of data points, recommendations, or other suggestions. The specific types of actionable insights can vary based on the jurisdiction(s) and associated laws, regulations, or other rules that govern the user and/or the process.

In further embodiments, the process 900 can identify clustering models (block 940). As described above, the clustering models can vary based on the application desired. Indeed, multiple clustering models may be identified in response to the type of digital model being processed.

In more embodiments, the process 900 can assign the digital model to a cluster associated with the clustering models (block 950). In some embodiments, the process 900 can process the digital model through multiple clustering models. Selecting multiple clusters may be done sequentially in response to certain results, or in parallel.

In various embodiments, the process 900 can generate a second set of actionable insights (block 960). In certain embodiments, the second set of actionable insights can be compared with the first set of actionable insights. In still further embodiments, the comparison of these actionable insights can account for one or more medical conditions, etc.

In still more embodiments, the process 900 can, in some optional embodiments, present a subset of first and second set of actionable insights (block 970). In certain embodiments, the process 900 can transfer these insights to other processes, methods, or devices for eventual presentation. In more embodiments, the process 900 can present actionable insights in response to one or more questions presented to the user prior to presentation, which can be utilized to cull, edit, or otherwise modify the presentation.

Although a specific embodiment for the process 900 for managing user data is discussed with respect to FIG. 9, any of a variety of systems and/or processes may be utilized in accordance with embodiments of the disclosure. For example, the process 900 may process the digital model with the clustering model prior to the classifier model, or may process them both in parallel. The elements depicted in FIG. 9 may also be interchangeable with other elements of FIGS. 1-8 and FIGS. 10-11 as required to realize a particularly desired embodiment.

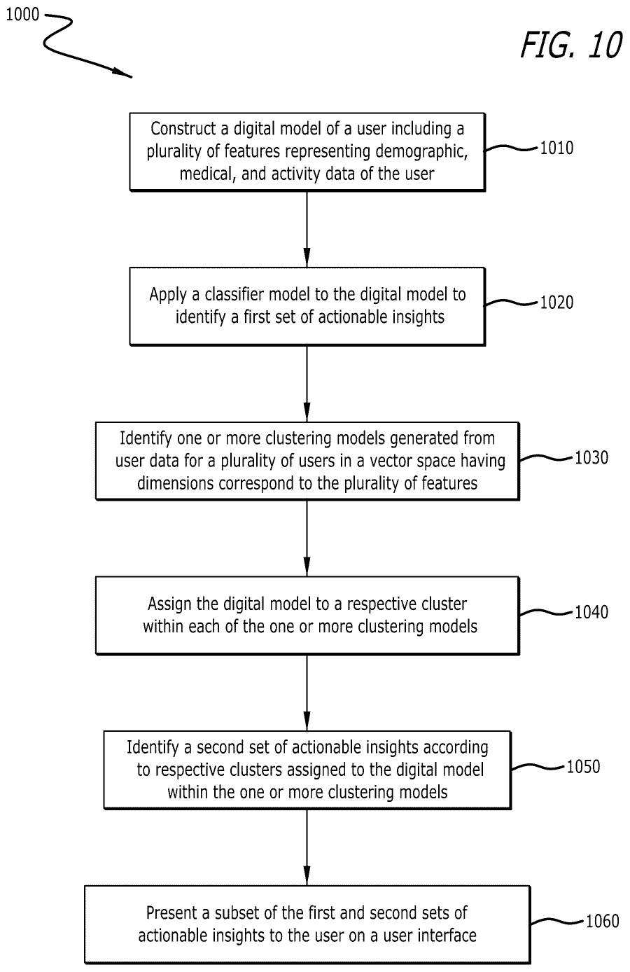

FIG. 10 is a flowchart of a more-detailed process 1000 of managing user data for a plurality of patient users, in accordance with some embodiments. In some embodiments, the method is applied at a server system. For convenience, the process 1000 is described as being implemented by a computer system (such as, for example, the computer system described within the embodiment shown in FIG. 2). In some embodiments, the process 1000 can be governed by instructions that are stored in a non-transitory computer readable storage medium and that are executed by one or more processors of the computer system. Each of the steps shown in FIG. 10 may correspond to instructions stored in computer memory or a non-transitory computer readable storage medium (e.g., the memory 214 of the computer system 200 in FIG. 2). The computer readable storage medium may include a magnetic or optical disk storage device, solid state storage devices such as Flash memory, or other non-volatile memory device or devices. The instructions stored on the computer readable storage medium may include one or more of: source code, assembly language code, object code, or other instruction format that is interpreted by one or more processors. Some operations in process 1000 may be combined and/or the order of some operations may be changed.

In many embodiments, the process 1000 can construct a digital model of a user including a plurality of features representing demographic, medical, and activity data of the user (block 1010). In additional embodiments, the process 1000 can apply one or more classifier models to the digital model to identify a first set of actionable insights (block 1020). In more embodiments, the process 1000 can additionally identify one or more clustering models generated from user data for a plurality of users in a vector space having dimensions correspond to the plurality of features (block 1030). In further embodiments, the process 1000 can assign the digital model to a respective cluster within each of the one or more clustering models (block 1040). In a number of embodiments, the process 1000 can identify a second set of actionable insights according to respective clusters assigned to the digital model within the one or more clustering models (block 1050). In still more embodiments, the process 1000 can present a subset of the first and second sets of actionable recommendations to the user on a user interface. (block 1060).

It should be understood that the particular order in which the operations in FIG. 10 have been described are merely exemplary and are not intended to indicate that the described order is the only order in which the operations could be performed. Additionally, it should be noted that details of other processes described above with respect to FIGS. 1-8 are also applicable in an analogous manner to process 1000 described above with respect to FIG. 10. For brevity, these details are not repeated here.

Although a specific embodiment for a more-detailed process 1000 for managing user data is discussed with respect to FIG. 10, any of a variety of systems and/or processes may be utilized in accordance with embodiments of the disclosure. For example, the process 1000 may avoid presenting the insights to the user itself and instead hand off that data to another process, method, or device to present. The elements depicted in FIG. 10 may also be interchangeable with other elements of FIGS. 1-9 and FIG. 11 as required to realize a particularly desired embodiment.

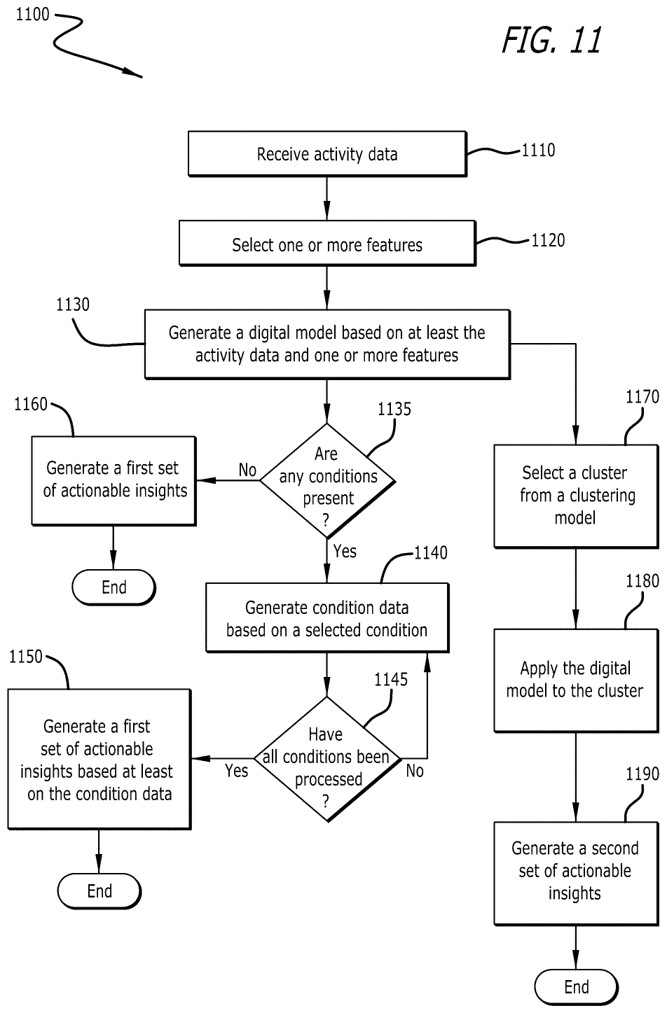

FIG. 11 is a flowchart of a process 1100 for generating actionable insights in some embodiments. In many embodiments, the process 1100 can receive activity data (block 1110). As discussed above, the activity data can be received from various sources such as health data, personal devices, and/or wearable devices, etc. The activity data can be collected from a specific moment, or may be configured to cover a period of time.

In additional embodiments, the process 1100 can select one or more features (block 1120). As described in more detail above, features can cover a variety of different aspects associated with a user. In some embodiments, each feature may be associated with a specific activity, demographic item, medical issue, etc.

In a number of embodiments, the process 1100 can generate a digital model based on at least the activity data and one or more features (block 1130). In some embodiments, the digital model may be configured based on the eventual cluster models, or classifier models. In these embodiments, the process 1100 can evaluate these models and the inputs required for processing, and select/format the activity data and/or features based on those evaluations.

In some embodiments, the process 1100 can determine if any conditions are present (block 1135). Often, these conditions are medical conditions associated with the user. In response to no conditions being present, the process 1100 can generate a first set of actionable insights (block 1160). This generation can often be done via one or more classifier models, such as those described above in previous figures.

However, when one or more conditions are present, the process 1100 can generate condition data based on a selected conditions (block 1140). This condition data can be formatted as an input into one or more classifier models. In additional embodiments, the condition data may be integrated into the digital model.

In various embodiments, the process 1100 can determine if all conditions have been processed (block 1145). If additional conditions still remain to be processed, then the process 1100 may again generate condition data based on the selected condition (block 1140). This process may repeat as many times as necessary until all conditions have been processed.

In response to all conditions being processed, the process 1100 can generate a first set of actionable insights based at least on the condition data (block 1150). As previously discussed, the condition data may be utilized as a separate input for a classifier or other similar module. However, the use of condition data may be done such to narrow down, filter out, or otherwise change the actionable insights generated.

In more embodiments, the process 1100 can select a cluster from a clustering model (block 1170). As discussed above, there are a plurality of different types of clustering models available, that may be suitable for various applications. The cluster selection may be done a single selection or may include a plurality of clusters that are processed sequentially or in parallel as desired.

In further embodiments, the process 1100 can apply the digital model to the cluster (block 1180). The application can be similar to the applications described above with respect to FIGS. 6A-7B. One or more clusters can be processed and may be done simultaneously or sequentially.

In still more embodiments, the process 1100 can generate a second set of actionable insights (block 1190). In certain embodiments, the second set of actionable insights can be compared against the first set of actionable insights for further processing. The processed set of first and second actional insights may be presented to a user in one or more formats.

Although a specific embodiment for a process 1100 for generating actionable insights is discussed with respect to FIG. 11, any of a variety of systems and/or processes may be utilized in accordance with embodiments of the disclosure. For example, the process 1100 may present or transmit the first and/or second actionable insights by themselves without processing them against each other. The elements depicted in FIG. 11 may also be interchangeable with other elements of FIGS. 1-10 as required to realize a particularly desired embodiment.

The terminology used in the description of the disclosure herein is for the purpose of describing particular embodiments only and is not intended to be limiting of the disclosure. As used in the description of the disclosure and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. Additionally, it will be understood that, although the terms “first,” “second,” etc. may be used herein to describe various elements, these elements should not be limited by these terms. These terms are only used to distinguish one element from another.

As used herein, the term “if” means “when” or “upon” or “in response to determining” or “in response to detecting” or “in accordance with a determination that,” depending on the context. Similarly, the phrase “if it is determined” or “if [a stated condition or event] is detected” means “upon determining” or “in response to determining” or “upon detecting [the stated condition or event]” or “in response to detecting [the stated condition or event]” or “in accordance with a determination that [a stated condition or event] is detected,” depending on the context.

The foregoing description, for purpose of explanation, has been described with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

Although various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages can be implemented in hardware, firmware, software or any combination thereof.

What is claimed is:

1. A method for managing data, comprising:
at a computer system having one or more processors and memory:
constructing a digital model of a user including a plurality of features representing demographic, medical, and activity data of the user;
applying a classifier model to the digital model to identify a first set of actionable insights;
identifying one or more clustering models generated from user data for a plurality of users in a vector space having dimensions correspond to the plurality of features;
assigning the digital model to a respective cluster within each of the one or more clustering models;
identifying a second set of actionable insights according to respective clusters assigned to the digital model within the one or more clustering models; and
presenting a subset of the first and second sets of actionable insights to the user on a user interface.

2. The method of claim 1, wherein the demographic, medical, and activity data of the user includes a subset of:
user-specified medical characteristics of the user;
historical medical data of the user, retrieved from a database of medical records of a medical institution;
activity data from one or more wearable devices worn by the user;
biomarker data measured by one or more laboratory tests of bodily fluids from the user; and
changes over time in the user-specified medical characteristics, the historical medical data, the activity data, and the biomarker data.

3. The method of claim 1, wherein the digital model has a predefined data structure in which the plurality of features is organized to be used as inputs to the classifier model and the one or more clustering models.

4. The method of claim 1, wherein the classifier model includes one or both of:
a plurality of first sub-classifier models, wherein each first sub-classifier model is configured to determine respective condition data of a plurality of predefined medical conditions; and
an activity classifier model that is configured to determine an activity style and an activity level of the user.

5. The method of claim 1, wherein applying the classifier model to the digital model to identify the first set of actionable insights further comprises:
generating a condition vector having a plurality of probability values corresponding to a plurality of predefined medical conditions; and
determining the first set of actionable insights based on the plurality of probability values corresponding to the plurality of predefined medical conditions.

6. The method of claim 5, wherein a lookup table associates a plurality of probability ranges of the plurality of predefined medical conditions with a plurality of predefined actionable insights, the method further comprising:
identifying a subset of the plurality of probability ranges for a subset of predefined medical conditions; and
based on the subset of probability ranges for the subset of predefined medical conditions, checking the lookup table to identify the first set of actionable insights from the plurality of predefined actionable insights.

7. The method of claim 5, wherein the plurality of predefined medical conditions includes a first medical condition corresponding to a first probability value, and the method further comprises comparing the first probability value to a first threshold to determine the first set of actionable insights.

8. The method of claim 7, wherein the plurality of predefined medical conditions includes a second medical condition corresponding to a second probability value, and the method further comprises comparing the second probability value to a second threshold to adjust the first set of actionable insights determined based on the first probability value of the first medical condition.

9. The method of claim 7, wherein the condition vector includes an activity level, and the method further comprises: based on the activity level, adjusting the first set of actionable insights determined based on the first probability value of the first medical condition.

10. The method of claim 5, wherein:
the plurality of predefined medical conditions include one or more of: asthma, heart disease, stroke, diabetes, arthritis, cancer, obesity, Alzheimer's disease, substance abuse, influenza, HIV, Zoonotic disease, tuberculosis, chronic kidney disease, or mental illness.

11. The method of claim 1, wherein applying the classifier model to the digital model to identify the first set of actionable insight comprises:
generating, by the classifier model, an action vector having a plurality of recommendation scores corresponding to a plurality of predefined actionable insights;
ranking the plurality of recommendation scores corresponding to the plurality of predefined actionable insights; and
selecting the first set of actionable insights having a highest recommendation score in the plurality of predefined actionable insights.

12. The method of claim 1, wherein the one or more clustering models includes a single clustering model corresponding to two or more clusters, each of which corresponds to one or more cluster recommendations.

13. The method of claim 12, wherein:
each of the two or more clusters corresponds to a respective one of a plurality of predefined medical conditions; and
the plurality of predefined medical conditions include one or more of: asthma, heart disease, stroke, diabetes, arthritis, cancer, obesity, Alzheimer's disease, substance abuse, influenza, HIV, Zoonotic disease, tuberculosis, chronic kidney disease, and mental illness.

14. A computer system, comprising:
one or more processors; and
memory storing one or more programs for execution by the one or more processors, the one or more programs further comprising instructions for:
constructing a digital model of a user including a plurality of features representing demographic, medical, and activity data of the user;
applying a classifier model to the digital model to identify a first set of actionable insights;
identifying one or more clustering models generated from user data for a plurality of users in a vector space having dimensions correspond to the plurality of features;
assigning the digital model to a respective cluster within each of the one or more clustering models; and
identifying a second set of actionable insights according to respective clusters assigned to the digital model within the one or more clustering models; and
presenting a subset of the first and second sets of actionable insights to the user on a user interface.

15. The computer system of claim 14, wherein each of the one or more clustering models corresponds to two or more clusters, each of which corresponds to one or more cluster recommendations, and the one or more programs further comprise instructions for:
determining the one or more cluster recommendations for each of the respective clusters assigned to the digital model within the one or more clustering models; and
consolidating the one or more cluster recommendations for each of the clusters assigned to the digital model to identify the second set of actionable insights.

16. The computer system of claim 15, wherein consolidating the one or more cluster recommendations for each of the clusters assigned further comprises:
resolving a conflict between (i) a first cluster recommendation of a first cluster of a first clustering model and (ii) a second cluster recommendation of a second cluster of a second clustering model.

17. The computer system of claim 14, wherein each of the one or more clustering models is implemented based on one or more of: a K-means clustering method, a density-based spatial clustering of applications with noise (DBSCAN) clustering method, a Gaussian Mixture Model method, balance iterative reducing and clustering using Hierarchies (BIRCH), an Affinity Propagation clustering method, a mean-shift clustering method, an ordering points to identify a clustering structure (OPTICS) method, and an agglomerative hierarchy clustering method.

18. A non-transitory computer-readable storage medium, storing one or more programs configured for execution by one or more processors, the one or more programs further comprising instructions for:
constructing a digital model of a user including a plurality of features representing demographic, medical, and activity data of the user;
applying a classifier model to the digital model to identify a first set of actionable insights;
identifying one or more clustering models generated from user data for a plurality of users in a vector space having dimensions correspond to the plurality of features;
assigning the digital model to a respective cluster within each of the one or more clustering models;
identifying a second set of actionable insights according to respective clusters assigned to the digital model within the one or more clustering models; and
presenting a subset of the first and second sets of actionable insights to the user on a user interface.

19. The non-transitory computer-readable storage medium of claim 18, wherein the one or more programs further comprise instructions for:
training the classifier model using first user data and corresponding ground truth; and
training the one or more clustering models using the first user data without the corresponding ground truth.

20. The non-transitory computer-readable storage medium of claim 18, wherein each of the first and second sets of actionable insights is one of: a medication with a dosage, an activity with an activity level, a food recipe, or a dietary supplemental with a suggested usage.

21. The non-transitory computer-readable storage medium of claim 18, wherein the one or more programs further comprise instructions for, prior to presenting the subset of the first and second sets of actionable insights:
identifying an inconsistency or a conflict between (i) one of the first set of actionable insights and (ii) one of the second set of actionable insights; and
resolving the inconsistency or the conflict by one of:
presenting a query and receiving a doctor response to the query;
automatically disabling both the one of the first set of actionable insights and the one of the second set of actionable insights; or
automatically, in accordance with a predefined rule, choosing one of the inconsistent or conflicting actionable insights.
